(19) United States
(12) Patent Application Publication
(10) Pub. No.: US 2023/0270805 A1
(43) Pub. Date: Aug. 31, 2023

(54) A COMPOSITION COMPRISING AN EXTRACT OF ALDER TREE OR THE ISOLATED COMPOUNDS THEREFROM FOR TREATING AND PREVENTING SKELETON MUSCLE-RELATED DISORDER AND THE USE THEREOF

(71) Applicant: Sookmyung Women's University Indusutry-Academic Cooperation Foundation, Seoul (KR)

(72) Inventors: Jae-Ha RYU, Seoul (KR); Hye Jin LEE, Seoul (KR); Sunghee CHOI, Suwon-si (KR)

(21) Appl. No.: 18/018,473

(22) PCT Filed: Aug. 3, 2021
(86) PCT No.: PCT/KR2021/010158

§ 371 Date: Jan. 27, 2023

(30) Foreign Application Priority Data
Aug. 11, 2020 (KR) 10-2020-0100747

(51) Int. Cl. A61K 36/185; A61K 31/216; A61K 31/12; A61K 31/7034; A61P 21/00; A61K 45/06

(52) U.S. Cl. CPC A61K 36/185; A61K 31/216; A61K 31/12; A61K 31/7034; A61P 21/00; A61K 45/06; A61K 2236/333

(57) ABSTRACT

The present invention related to a composition comprising an extract of alder tree or the isolated compounds therefrom for treating and preventing skeleton muscle disease and the use thereof. It has been confirmed that the inventive extract/compounds showed potent improving or treating effect on muscle loss through various in vitro test and animal model tests, for example, Stimulation effect of inventive extract/compounds on the initiation effect of myoblast differentiation (Experimental Example 1, 3 and 4), confirming that inventive extract/compounds increases the number of cylinder-shaped multinucleated myotubes, resulting in promoting muscle cell differentiation; Study on p38 MAPK signaling system mechanism of inventive extract/compounds for promoting myoblast differentiation (Experimental Example 2 and 5); Inhibitory effect of inventive extract/compounds on muscle loss (Experimental Example 6 and 7); improving effect on motility in animal model of muscle loss (In vivo) (Experimental Example 8) confirming that s that inventive extract/compounds promotes muscle cell differentiation as well as improves motor capacity decline and muscle loss. Therefore, the inventive extract/compounds of the present invention can be usefully used in a pharmaceutical composition, health functional food, and health supplement food for preventing or treating on skeleton muscle disease.

### TECHNICAL FIELD

The present invention is related to a composition comprising an extract of alder tree or the isolated compounds therefrom for treating and preventing skeleton muscle disease and the use thereof.

### BACKGROUND ART

The present invention is related to a composition comprising an extract of alder tree or the isolated compounds therefrom for treating and preventing skeleton muscle disease and the use thereof.

Muscles can be divided into the following three types in terms of structure and function: (1) skeletal muscles that are located directly under the skin of the hands, feet, chest, belly, and back, and are attached to the bones between the bones, (2) the myocardium that forms the heart wall, and (3) the visceral muscles that form the walls of the stomach, bladder, uterus, etc.

There has been reported that the purpose of studies on muscle regeneration is focusing on recovering various skeletal muscle degenerative diseases such as atony, muscular atrophy, muscular dystrophy, muscle dystrophy, myotonia, amyotrophic lateral sclerosis (ALS), myasthenia gravis, cachexia, sarcopenia and the like (J Cachexia Sarcopenia Muscle. 2015, 6:197)

Progressive muscle weakness and the loss of its function threatens quality of life and reduces the survival rates in cancer patients. More than 30% of cancer patients die due to their weight loss caused by weight loss. Such loss of muscle function is commonly accompanied by myo-proteins denaturation and decreased cross-sectional area of muscle fiber, as well as decreased muscle strength, nuclear number of myofibers and insulin responsiveness etc.

In addition to cancer, muscle loss can also be caused by the progression of aging and various chronic diseases. As aging progresses, some of the newly regenerating skeletal muscle is replaced by fibrous tissue, resulting in sarcopenia, in which the body's skeletal muscle mass and strength are reduced. Muscle loss also occurs in the old patients suffering with hypertension, impaired glucose tolerance, and chronic diseases such as diabetes, obesity, dyslipidemia, atherosclerosis, and cardiovascular disease etc (Pharmacol Res. 2015, 99:86).

Therapeutic strategies for muscle degeneration include the inhibition of inflammatory molecules and myostatin or the activation of cyclic AMP, PGC (proliferator-activated receptor gamma coactivator)-1α and insulin signaling system. Despite the number of potential drugs being developed, megestrol acetate has been reported as the only treating agent for muscular dystrophy approved by the U.S. Food and Drug Administration. Drugs for the treatment and prevention of muscle degeneration have been reported to exhibit the inhibiting activity from muscle protein catabolism or enhancing activity of satellite cell functions. (Pharmacol. Res. 2015, 99:86).

The anabolic action and catabolism in muscle are balanced and muscle regeneration is regulated, and within muscle cells, several vital signaling processes are regulated in the pathway. When a signaling response is activated that induces muscle synthesis rather than the breakdown of muscle proteins, the muscle protein synthesis is increased, leading to an increase in muscle size (hypertrophy) or an increase in the number of muscle fibers (hyperplasia), resulting in excessive muscle reproduction. (Scientific Reports 2016, 6:31142).

Muscle-regeneration inducing factors induce the synthesis of muscle protein synthesis by activating the PH3K (phosphatidylinositol-3 kinase)/Akt pathway in muscle cells and phosphorylating downstream proteins accordingly. Among them, the activity of mTOR (mammalian target of rapamycin) by PI3K/Akt signaling is recognized as a central growth signaling mechanism that integrates various growth signals in cells. When mTOR is activated, two downstream targets, 4EBP1 (4E-binding protein) and p70S6K (phosphorylated 70-kDa ribosomal S6 kinase), are activated, thereby inducing muscle protein synthesis and increasing muscle mass. (Scientific Reports 2016, 6:31142, J Biol. Chem. 2003, 78:40717).

There has been reported that the differentiation of myocytes and muscle regeneration are regulated by various factors in addition to mTOR. (Cell Mol. Life Sci. 2013, 70: 4117). Among them, myoD initiates the expression of specific genes involved in muscle differentiation, leading to the differentiation of mesenchymal stem cells into myoblasts. Myogenin regulated by MyoD and MHC (myosin heavy chain) induce the fusion of myoblasts to form myotubes and muscle fibers. The muscle fibers formed through the process, form bundles and finally form muscle. (Cell Mol. Life Sci. 2013, 70:4117; Sci Signal. 2013, 6:272).

Under normal conditions, quiescent satellite cells as primitive stem cells continuously proliferate and differentiate. Damaged muscles secrete a variety of growth factors that activate the proliferation of myogenic satellite cells, referred to as myoblast. Activated myoblasts induce various myogenic factors such as Myo D, Myf (myogenic factor)-5, myogenin and MRF-4.

MyoD and Myf-5, transcription factors specifically expressed in the myogenic lineage and play an important role in initiating myoblast differentiation. (J. Biol. Chem. 2002, 277:49831).

In particular, Myo D induces the expression of myotropic proteins such as MHC and myogenin through binding with non-muscular specific factors such as E protein, Mef-2 family proteins, and transcription cofactors etc.

Alnus japonica steud is a deciduous tree belonging to the birch family (Betulaceae) and distributed throughout Korea except South Chungcheong Province. There have been reported that the plant contains various ingredients such as lupenone, glutenol, taraxerol, sitosterol, heptacosane etc and it has also known to be effective in treating various diseases such as enteritis, diarrhea, traumatic bleeding, etc. (B. S. CHUNG et al., Dohaehyangyak Daesajeon, Youngrim-press, p802, 1990).

The present inventors have been tried to develop the effective treating agent to treat various skeleton muscle degeneration disease such as myasthenia, cachexia and sarcopenia from natural resource till now. For example, Korea patent registration No. 10-2022279 B1 discloses the composition comprising an extract of Angelica keiskei for treating or preventing muscle-related disorder; Korea patent registration No. 10-2132126 B1 discloses the composition comprising 4-hydroxyderricin or xanthoangelol isolated from an extract of Angelica keiskei for treating and preventing cachexia or sarcopenia; Korea patent registration No. 10-2040119 B1 discloses the composition comprising the isolated compounds from an extract of Angelica keiskei for treating and preventing muscle-related disorder; Korea patent registration No. 10-1965699 B1 discloses the composition comprising an extract of Allium sativum L. for treating and preventing muscle-related disorder); Korea patent registration No. 10-2002260 B1 discloses the composition comprising the compounds isolated from an extract of Allium sativum L. for treating and preventing muscle-related disorder); Korea patent registration No. 10-1996184 B1 discloses an Adjuvant of anti-cancer agent comprising and an extract of Allium sativum L. for treating and preventing cachexia; Korea patent publication No. 10-2018-0131770 discloses the composition comprising an extract of Coptis chinensis F. for treating and preventing muscle-related disorder etc till now.

However, there has been not reported or disclosed on the therapeutic effect on skeleton muscle disease of an extract of alder tree or the compounds isolated therefrom as an active ingredient in any of above cited literatures, the disclosures of which are incorporated herein by reference.

To investigate a treating effect of an extract of alder tree or the compounds isolated therefrom on skeleton muscle disease, the inventors of the present invention have intensively carried out various in vitro test and animal model tests, for example, Stimulation effect of inventive extract/compounds on the initiation effect of myoblast differentiation (Experimental Example 1, 3 and 4), confirming that inventive extract/compounds increases the number of cylinder-shaped multinucleated myotubes, resulting in promoting muscle cell differentiation; Study on p38 MAPK signaling system mechanism of inventive extract/compounds for promoting myoblast differentiation (Experimental Example 2 and 5); Inhibitory effect of inventive extract/compounds on muscle loss (Experimental Example 6 and 7); improving effect on motility in animal model of muscle loss (In vivo) (Experimental Example 8) confirming that the inventive extract/compounds promotes muscle cell differentiation as well as improves motor capacity decline and muscle loss. Therefore, the inventive extract/compounds of the present invention can be usefully used in a pharmaceutical composition, health functional food, and health supplement food for preventing and treating on skeleton muscle disease.

These and other objects of the present invention will become apparent from the detailed disclosure of the present invention provided hereinafter.

### DISCLOSURE

### Technical Problem

The present inventors have technical object to develop effective natural product for treating or improving skeletal muscle diseases as a technical task.

### Technical Solution

Accordingly, it is an object of the present invention to provide a pharmaceutical composition comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the treatment or prevention of skeletal muscle diseases.

It is an another object of the present invention to provide a health functional food comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

The term “alder tree” disclosed herein, comprises all the extraction source of the plants belonged to the same genus, such as root part, stem part, bark part, xylem part, herb part, leaf part of Alnus japonica steud, Alnus japonica var. koreana and the like, preferably, stem part including root, bark and xylem part of Alnus japonica steud or Alnus japonica var. koreana

The term “an extract of alder tree” disclosed herein, comprises “crude extract”, and purified “non-polar solvent soluble extract” or non-polar solvent soluble extract of alder tree.

The term “crude extract of alder tree” disclosed herein, comprises the crude extract which can be dissolved in any polar solvent, for example, distilled water, spirit, lower alcohols such as methanol, ethanol, butanol and the like, or the mixtures thereof, preferably, spirit or the mixture of water and ethanol or spirit, more preferably, spirit or 30-100% ethanol or spirit etc.

The term “non-polar solvent soluble extract of alder tree” disclosed herein, comprises “the remaining purified non-polar solvent soluble extract after fractionating the crude extract with non-polar solvent”, which can be soluble in any non-polar solvent, for example, n-hexane, chloroform, methylene chloride, ethylacetate or the mixtures thereof, etc.

The term “polar solvent soluble extract of alder tree” disclosed herein, comprises “the remaining purified polar solvent soluble extract after removing non-polar solvent soluble extract from the crude extract, which can be soluble in any polar solvent, for example, butanol, methanol, water or the mixtures thereof, etc.

The term “the inventive extract/compounds of alder tree” disclosed herein can be analyzed with HPLC (High performance liquid chromatography) using by mobile phase, for example, the mixture of acidic water solution with methanol or non-polar solvent such as acetonitrile.

The term “skeletal muscle diseases” disclosed herein, comprises “one or more skeletal muscle diseases selected from the group consisting of muscular atony, muscular atrophy, muscular dystrophy, muscle degeneration, muscle stiffness, amyotrophic axonal sclerosis, myasthenia gravis, cachexia and sarcopenia, specifically, skeletal muscle related diseases due to senile muscular atrophy or cancer disease, more specifically, muscular atrophy, muscular dystrophy, muscle degeneration, muscle stiffness, amyotrophic axonal sclerosis, myasthenia gravis, cachexia, sarcopenia, and muscle loss due to senile muscular atrophy or cancer disease.

It is an another object of the present invention to provide a preventing or treating agent of skeletal muscle diseases or anti-cancer adjuvant comprising an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

It is an another object of the present invention to provide a pharmaceutical composition or anti-cancer adjuvant comprising the combination of an extract of alder tree or the compounds isolated therefrom; and exiting anti-cancer drugs as an active ingredients for the improvement or prevention of skeletal muscle diseases.

It is an another object of the present invention to provide a health functional food comprising the combination of an extract of alder tree or the compounds isolated therefrom; and exiting anti-cancer drug(s) as an active ingredients for the improvement or prevention of skeletal muscle diseases.

The term “exiting anti-cancer drug(s)” disclosed herein comprises Cyclophosphamide, methotrexate, 5-fluorouracil, Doxorubicin, Mustine, vincristine, procarbazine, prednisolone, bleomycin, vinblastine, dacarbazine, etoposide, cisplatin, Epirubicin, cisplatin, capecitabine, oxaliplatin and the like, which are used for treating various cancer diseases at present.

The term “cancer disease(s)” disclosed herein comprises leukemia, lymphoma, myeloma, myelodysplastic syndrome, breast cancer, head and neck cancer, esophageal cancer, stomach cancer, colon cancer (=colon carcinoma), rectal cancer, anal cancer, hepatocellular liver cancer, cholangiocarcinoma, gallbladder cancer, pancreatic cancer, lung cancer (non-small cell lung cancer, small cell lung cancer), thymic cancer, kidney cancer, bladder cancer, prostate cancer, testicular cancer, ovarian cancer, cervical cancer, sarcoma, gastrointestinal organic tumor (GIST), primary site unknown cancer, mesothelioma, melanoma, neuroendocrine tumor, skin cancer, blood cancer etc.

Hereinafter, the present invention is described in detail.

The inventive extract/compounds of alder tree can be prepared in detail by following procedures,

The inventive crude extract of alder tree can be prepared by follows; For example, alder tree is dried, cut, crushed and mixed with distilled water, spirit, lower alcohols such as methanol, ethanol, butanol and the like, or the mixtures thereof, preferably, spirit or the mixture of water and ethanol or spirit, more preferably, spirit or 30-100% ethanol; the solution is extracted at the temperature ranging from 30 to 150° C., preferably from 50 to 100° C., for the period ranging from 30 mins to 48 hours, preferably, 6 hours to 36 hours, with extraction method by the extraction with hot water, cold water, reflux extraction, or ultra-sonication extraction, preferably, cold water extraction with 1 to 20 times, preferably 2 to 10 times, consecutively; the residue is filtered to obtain the supernatant to be concentrated with rotary evaporator, at the temperature ranging from 20 to 100° C., preferably from 50 to 70° C. and then dried to obtain dried crude extract powder of alder tree.

Additionally, polar solvent soluble and non-polar solvent soluble extract of present invention can be prepared by following procedure; the crude extract prepared by above step, is suspended in water, and then is mixed with 0.0005 to 500-fold, preferably, 0.05 to 5-fold volume (v/w %) of non-polar solvent such as n-hexane, chloroform, methylene chloride, ethylacetate and the like; the non-polar solvent soluble layer is collected to obtain non-polar solvent soluble extract of the present invention and the remaining polar solvent soluble layer is further fractionated into butanol and water layer to obtain polar solvent soluble extract of the present invention which is soluble in water, lower alcohols such as butanol, methanol, or the mixtures thereof according to the conventional fractionation process well known in the art.

The inventive compounds of alder tree can be prepared in detail by following procedures,

The inventive compounds of alder tree can be prepared by follows; For example, the inventive crude extract of alder tree can be fractionated into non-polar soluble extract such as chloroform and the non-polar soluble extract is further subject to at least one purification process selected from (i) reverse phase partition chromatography, (ii) flash column chromatography, (iii) RP C18 column chromatography, (iv) Silica gel column chromatography, (v) ion exchange chromatography or (iv) size exclusion chromatography repeatedly, preferably, repeated Silica gel column chromatography method using mobile phase such as running solvent system varying with their polarity, starting with 100% n-hexane and increasing their polarity with polar acetone solvent to afford inventive compounds of the present invention.

To investigate a treating effect of an extract of alder tree or the compounds isolated therefrom mentioned above on skeleton muscle disease, the inventors of the present invention have intensively carried out various in vitro test and animal model tests, for example, Stimulation effect of inventive extract/compounds on the initiation effect of myoblast differentiation (Experimental Example 1, 3 and 4), confirming that inventive extract/compounds increases the number of cylinder-shaped multinucleated myotubes, resulting in promoting muscle cell differentiation; Study on p38 MAPK signaling system mechanism of inventive extract/compounds for promoting myoblast differentiation (Experimental Example 2 and 5); Inhibitory effect of inventive extract/compounds on muscle loss (Experimental Example 6 and 7); improving effect on motility in animal model of muscle loss (In vivo) (Experimental Example 8) confirming that s that inventive extract/compounds promotes muscle cell differentiation as well as improves motor capacity decline and muscle loss. Therefore, the inventive extract/compounds of the present invention can be usefully used in a pharmaceutical composition, health functional food, and health supplement food for preventing or treating on skeleton muscle disease.

It is an another object of the present invention to provide a pharmaceutical composition or health functional food comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) prepared by the above-described preparation method as an active ingredients for the treatment or prevention of skeletal muscle diseases.

The inventive compound can be transformed into their pharmaceutically acceptable salt and solvates by the conventional method well known in the art. For the salts, acid-addition salt thereof formed by a pharmaceutically acceptable free acid thereof is useful and can be prepared by the conventional method. For example, after dissolving the compound in the excess amount of acid solution, the salts are precipitated by the water-miscible organic solvent such as methanol, ethanol, acetone or acetonitrile to prepare acid addition salt thereof and further the mixture of equivalent amount of compound and diluted acid with water or alcohol such as glycol monomethylether, can be heated and subsequently dried by evaporation or filtrated under reduced pressure to obtain dried salt form thereof.

As a free acid of above-described method, organic acid or inorganic acid can be used. For example, organic acid such as methansulfonic acid, p-toluensulfonic acid, acetic acid, trifluoroacetic acid, citric acid, maleic acid, succinic acid, oxalic acid, benzoic acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid and the like, and inorganic acid such as hydrochloric acid, phosphoric acid, sulfuric acid, nitric acid, tartaric acid and the like can be used herein.

Further, the pharmaceutically acceptable metal salt form of inventive compounds may be prepared by using base. The alkali metal or alkali-earth metal salt thereof can be prepared by the conventional method, for example, after dissolving the compound in the excess amount of alkali metal hydroxide or alkali-earth metal hydroxide solution, the insoluble salts are filtered and remaining filtrate is subjected to evaporation and drying to obtain the metal salt thereof. As a metal salt of the present invention, sodium, potassium or calcium salt are pharmaceutically suitable and the corresponding silver salt can be prepared by reacting alkali metal salt or alkali-earth metal salt with suitable silver salt such as silver nitrate.

The pharmaceutically acceptable salt of the compound comprises all the acidic or basic salt which may be present at the compounds, if it does not indicated specifically herein. For example, the pharmaceutically acceptable salt of the present invention comprise the salt of hydroxyl group such as the sodium, calcium and potassium salt thereof; the salt of amino group such as the hydrogen bromide salt, sulfuric acid salt, hydrogen sulfuric acid salt, phosphate salt, hydrogen phosphate salt, dihydrophosphate salt, acetate salt, succinate salt, citrate salt, tartarate salt, lactate salt, mandelate salt, methanesulfonate (mesylate) salt and p-toluenesulfonate (tosylate) salt etc, which can be prepared by the conventional method well known in the art.

The pharmaceutical composition for treating purposed diseases could contain about 0.01 to 99 w/w % the above extract/compounds of the present invention based on the total weight of the composition.

However, the amount and each component of the above-mentioned composition can be varied with the patient's condition, development of patient's disease, the sort of disease etc.

The inventive composition comprising the above extract/compounds of the present invention may additionally comprise conventional carrier, adjuvants or diluents in accordance with a using method.

The inventive composition according to the present invention can be formulated in oral dosage form such as powder, granule, tablet, capsule, suspension, emulsion, syrup, aerosol and the like; topical preparation; or injection solution. The inventive composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, magnesium stearate and mineral oil. The formulations may additionally include excipients such as fillers, bulking agents, binders, wetting agents, disintegrating agents, surfactants, diluents and the like. The solid oral dosage form comprises tablet, pill, powder, granule, capsule and the like and the solid oral dosage form is prepared by adding at least one excipient such as starch, calcium carbonate, sucrose, lactose or gelatin and the like to the inventive protein. Lubricant such as magnesium stearate or talc may be used. The aqueous oral dosage form comprises suspension, oral solution, emulsion, syrup and the aqueous oral dosage form may comprise several excipients such as wetting agents, sweetener flavoring agents, preservatives, as well as water, liquid paraffin. The parenteral dosage form comprises sterilized aqueous solution, non-aqueous solvent, suspension, emulsion, lyophilized preparation, suppository, and the like. Suitable examples of the carriers include propylene glycol, polyethylene glycol, vegetable oils such as olive oil, injectable ester such as ethyl oleate. Base for suppository may include witepsol, macrogol, tween 61, cacao butter, laurin, glycerogelatine etc., but are not limited to them.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01 mg/kg to 10 g/kg, preferably, 1 mg/kg to 1 g/kg by weight/day of the inventive composition of the present invention. The dose may be administered in a single or multiple doses per day.

The pharmaceutical composition of present invention can be administered to a subject animal such as mammals (rat, mouse, domestic animals or human) via various routes. All modes of administration are contemplated, for example, administration can be made orally, rectally or by intravenous injection.

It is an another object of the present invention to provide a treating agent of cachexia or senile muscular atrophy comprising an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) as an active ingredients. The treating agent is used for the effect of improving weight change or appetite recovery in combination therapy of senile muscular atrophy or cachexia and provides a combination therapy using the composition of the present invention rather than administration of anticancer drugs alone in cachexia treatment.

It is an another object of the present invention to provide a treating agent of senile muscular atrophy or cachexia comprising the combination of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6); and exiting anti-cancer drugs as an active ingredients for the improvement or prevention of skeletal muscle diseases.

It is an another object of the present invention to provide an anti-cancer adjuvant comprising an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) as an active ingredients.

It is an another object of the present invention to provide an anti-cancer adjuvant comprising the combination of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6); and exiting anti-cancer drugs as an active ingredients.

It is an object of the present invention to provide a method of treating or preventing skeletal muscle diseases in a mammal comprising administering to said mammal an effective amount of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6), together with a pharmaceutically acceptable carrier thereof.

It is an object of the present invention to provide a use of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) for the manufacture of therapeutic agent for the treatment and prevention of skeletal muscle diseases in human or mammal.

It is an another object of the present invention to provide a health functional food comprising an extract of Alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

It is an another object of the present invention to provide a health functional food comprising the combination of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6); and exiting anti-cancer drugs as an active ingredients for the improvement or prevention of skeletal muscle diseases.

The term “a health functional food” defined herein comprises the functional food having enhanced functionality such as physical functionality or physiological functionality by adding the inventive protein of the present invention to conventional food to prevent or improve the purposed diseases in human or mammal and stipulated by the Law for Health Functional Foods 6727 in Republic of Korea.

The health functional food composition for preventing and improving purposed diseases could contain about 0.01 to 95 w/w %, preferably 1 to 80 w/w % of the inventive composition of present invention based on the total weight of the composition.

Moreover, the inventive protein of the present invention also can be used as a main component or additive and aiding agent in the preparation of various functional health food and health supplement food for the prevention or improvement of a purposed treating disease.

The inventive health functional food may be prepared and processed by the form of pharmaceutically acceptable dosage form such as powder, granule, tablet, capsule, pills, suspension, emulsion, syrup and the like; or the functional health food form such as tea bag, leached tea, health beverage type and the like.

It is an another object of the present invention to provide a health supplement food comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

It is an another object of the present invention to provide a health supplement food comprising the combination of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6); and exiting anti-cancer drugs as an active ingredients for the improvement or prevention of skeletal muscle diseases.

It is an another object of the present invention to provide a food or food additive comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

It is an another object of the present invention to provide a food or food additive comprising the combination of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6); and exiting anti-cancer drugs as an active ingredients for the improvement or prevention of skeletal muscle diseases.

The above-mentioned term “as a main component” means that the above health supplement food comprises about 30 to 99 (w/w %), preferably 50 to 99 (w/w %), more preferably 70 to 99 (w/w %) of the inventive protein of present invention based on the total weight of the composition.

When the inventive extract/compound of the present invention is used as a component in the health functional beverage composition, the health functional beverage composition can comprise other component such as flavoring agent or natural carbohydrate without limits like that typical beverage composition. Examples of the natural carbohydrate comprise monosaccharide such as glucose, fructose etc; disaccharide such as maltose, sucrose etc; and polysaccharide, for example, sugar such as dextrin, cyclodextrin, and sugar alcohol such as xylitol, sorbitol, erythritol. Natural flavoring agent (thaumatin, stevia extract (rebaudioside A, glycyrrhizin, etc)) and synthetic flavoring agent (saccharin, aspartame, etc) may be added in the health functional beverage composition. The amount of natural carbohydrate generally ranges from about 1 to 20 g, preferably about 5 to 12 g per 100 ml of the present composition.

When the inventive extract/compound of the present invention is used as a food additive of the health food, the combined herb extract may be added intact or used with other food ingredient according to general process. Examples of the food comprises meat products, sausage, bread, chocolate, candy, snack, cracker, biscuit, pizza, ramen, noodle products, chewing gum, dairy products such as ice cream, soup, beverage, tea, drinks, alcohol drink, vitamin complex etc, but not intended herein to limit thereto, for preventing or improving of purposed disease.

The other components than the aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Also, the above-described protein can be added to food or beverage for prevention and improvement of purposed disorder. The amount of the above-described protein in food or beverage as a functional health food or health supplement food may generally range from about 0.01 to 15 w/w % of total weight of food for functional health food composition. And the protein of the present invention may be added 0.02 to 5 g, preferably 0.3 to 1 g per 100 ml in health beverage composition.

### Advantageous Effects

The inventors of the present invention confirmed the treating effect of an extract of alder tree or the compounds isolated therefrom mentioned above on muscle-related disorder through various in vitro test and animal model tests, for example, stimulation effect of inventive extract/compounds on the initiation effect of myoblast differentiation (Experimental Example 1, 3 and 4), confirming that inventive extract/compounds increases the number of cylinder-shaped multinucleated myotubes, resulting in promoting muscle cell differentiation; Study on p38 MAPK signaling system mechanism of inventive extract/compounds for promoting myoblast differentiation (Experimental Example 2 and 5); Inhibitory effect of inventive extract/compounds on muscle loss (Experimental Example 6 and 7); improving effect on motility in animal model of muscle loss (In vivo) (Experimental Example 8) confirming that the inventive extract/compounds promotes muscle cell differentiation as well as improves motor capacity decline and muscle loss. Therefore, the inventive extract/compounds of the present invention can be usefully used in a pharmaceutical composition, health functional food, and health supplement food for preventing and treating on skeleton muscle disease.

### DESCRIPTION OF DRAWINGS

FIG. 1 depicts the stimulation effect of inventive extract on MHC and myoD expression;

FIG. 2 shows the effect of inventive extract on MHC expression in the number of cylinder-shaped multinucleated myotubes;

FIG. 3 presents the effect of inventive extract on p38 MAPK signaling system mechanism;

FIG. 4 represents the stimulation effect of inventive compounds 1-6 on MHC expression;

FIG. 5 depicts shows the effect of inventive compounds 1-6 on MHC expression in the number of cylinder-shaped multinucleated myotubes;

FIG. 6 shows the stimulation effect of inventive compound 1 on MHC and myoD expression;

FIG. 7 presents the effect of inventive compound 1 on MHC expression in the number of cylinder-shaped multinucleated myotubes;

FIG. 8 represents the effect of inventive compound 1 on p38 MAPK signaling system mechanism;

FIG. 9 depicts the effect of inventive extract on MHC and MAFbx expression in the myotube cell treated with dexamethasone to indue muscle loss;

FIG. 10 shows the stimulation effect of inventive extract on MHC expression of cylinder-shaped multinucleated myotubes in the myotube cell treated with dexamethasone to indue muscle loss;

FIG. 11 shows the stimulation effect of inventive compounds 1-6 on MHC expression of cylinder-shaped multinucleated myotubes in the myotube cell treated with dexamethasone to indue muscle loss.

### BEST MODE

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Examples

The following Examples and Experimental Examples are intended to further illustrate the present invention without limiting its scope.

### Example 1. Preparation of a Crude Extract of Alnus japonica Steud

3 L of 100% ethanol (spirit) was added to 600 g of the stem including the bark and xylem of the dried Alnus japonica steud (Yeongcheon, North Gyeongsang Province, Korea) and extracted at room temperature for 12 hours to extracted solution. The solution was filtered and concentrated under reduced pressure conditions. The concentrated ethanol (spirit) extract was lyophilized in a freeze dryer (ScanVac, Labogene) under reduced pressure conditions to obtain 28 g of dried powder of crude extract of Alnus japonica steud (hereinafter referred to as AJE) and used in the next experiment.

### Example 2. Preparation of a Purified Fractions of Alnus japonica Steud

28 g of dried powder of crude extract of Alnus japonica steud obtained in Example 1 is suspended by adding 500 mL of purified water, 500 mL of n-hexane is added to the suspension to fractionate into n-hexane soluble fraction. 500 mL of chloroform, 500 mL of ethyl acetate and 500 mL of butanol are sequentially added to the remaining water suspension to afford each fraction. Each fraction was dried under reduced pressure respectively to afford 4.1 g of n-hexane soluble fraction (hereinafter referred to as AJH), 2.5 g of chloroform soluble fraction (hereinafter referred to as AJC), 2.9 g, and ethyl acetate soluble fraction (hereinafter referred to as AJE) and 1.8 g of butanol soluble fraction (hereinafter referred to as AJB), respectively.

### Example 3. Isolation of Inventive Compounds from Ration of an Extract of Alnus japonica Steud

In order to find active ingredient from an extract of Alnus japonica steud obtained in the above, 2.5 g of chloroform soluble fraction (AJC) was further purified with silica gel column chromatography using mixture solvent system (n-hexane/acetone gradient system, 50:1→1:2) to afford 3 subfractions (CF1, CF2 and CF3) and the CF2 subfraction was further purified with reversed-phase column chromatography using mixture solvent system (MeOH gradient system, 50%→100%) to afford compound 1 ((−)-(2R,3R)-1,4-O-diferuloylsecoisorriscinol {(−)-(2R,3R)-1,4-O-diferuloylsecoisolariciresinol (DFS) compound 1, 23 mg).

2.9 g of ethyl acetate soluble fraction was further purified with silica gel column chromatography using by chloroform/methanol gradient method to obtain 13 fractions (EF1˜EF13). The EF3 fraction was further purified with silica gel column chromatography using by hexane/ethyl acetate solvent conditions to obtain 7 subfractions (EF1-EF7) and the EF3-3 subfraction was further purified with silica gel column chromatography using hexane/acetone solvent conditions to afford compound 2 (10.9 mg).

The EF8 fraction was further purified with silica gel column chromatography using by dichloromethane/methanol solvent conditions to afford 3 subfractions (EF8-1˜3) and the EF8-2 subfraction was again purified with RP-MPLC under 10%-100% methanol solvent conditions to afford several subfractions. the EF8-2-2 among the subfractions was further purified with HPLC Prep. Using by 50% methanol solvent condition to obtain compound 3 (9.5 mg). The EF10 fraction was further purified with MPLC using by 10% to 100% acetonitrile solvent condition and then HPLC prep under 50% methanol to afford compound 4 (8.5 mg), compound 5 (15 mg) and compound 6 (8.6 mg).

Compound 1 was identified as (−)-(2R,3R)-1,4-O-diferuloylsecoisolariciresinol (DFS, a lignan derivative) with more than 95% purity by comparing the physicochemical data of HPLC and ¹H-NMR with those disclosed in previous literature (J. Chem. Soc. Chem. Commun., 1975, 9:316).

Compounds 2, 3, 4, 5, and 6 were identified as diarylheptanoid derivatives, platyphyllenone (compound 2: Chem. Pharm. Bull. 1996, 44:1033), (5R)—O-methylhirsutanonol (compound 3: Chem. Pharm. Bull. 2006, 54:139), hirsutanonol (compound 4: J. Nat. Prod., 1998, 61:1292), platyphylloside (compound 5: Phytochem. 1993, 32:365), oregonin (compound 6: Chem Pharm Bull., 2010, 58: 238), respectively, by comparing the physicochemical data of HPLC and ¹H-NMR with those disclosed in previous literatures.

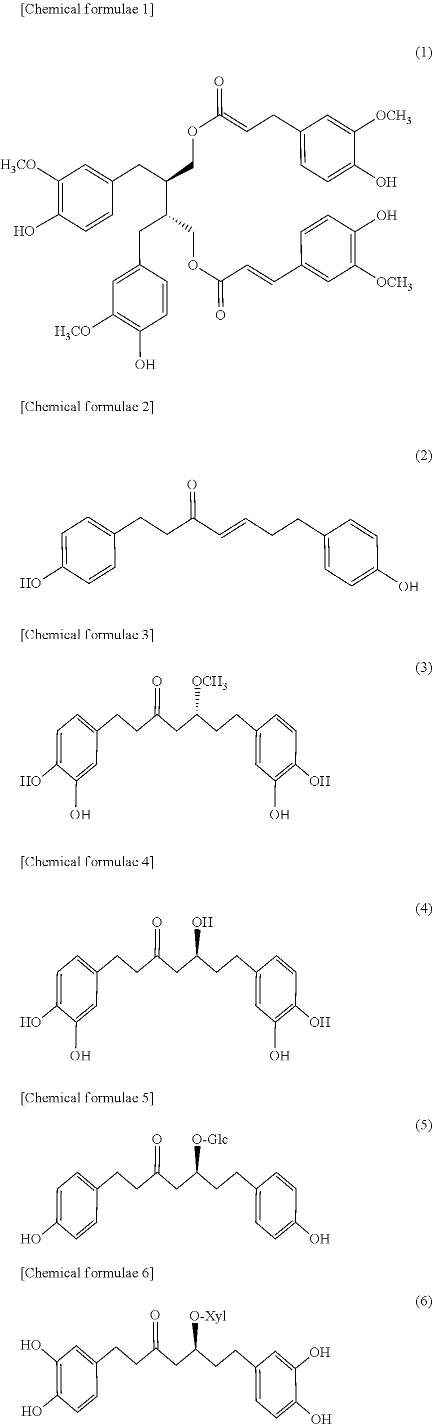

(A) Compound 1: (−)-(2R,3R)-1,4-O-diferuloylsecoisolariciresinol (DFS)

appearance: white powder

Molecular formulae: C₄₀H₄₂O₁₂ (MW. 714.27);

¹H-NMR (DMSO-d₆, 400 MHz): δ9.59 (1H, brs, —OH), 8.69 (1H, brs, —OH), 7.52 (2H, d, J=15.8 Hz, H-7″, 7″′), 7.29 (2H, d, J=1.9 Hz, H-2″, 2″′), 7.09 (2H, dd, J=8.3, 1.9 Hz, H-6″, 6″′), 6.77 (2H, d, J=8.1 Hz, H-5″, 5″′), 6.66 (2H, s, J=1.4 Hz, H-2, 2′), 6.64 (2H, s, H-5, 5′), 6.53 (2H, dd, J=8.0, 1.8 Hz, H-6, 6′), 6.48 (1H, s, J=15.9 Hz, H-8″′), 6.46 (1H, d, J=15.9 Hz, H-8″), 4.26 (2H, dd, J=11.2, 6.4 Hz, H-9), 4.07 (2H, dd, J=11.3, 5.0 Hz, H-9′), 3.80 (3H, s, —OCH₃), 3.67 (3H, s, —OCH₃), 2.75 (2H, dd, J=13.6, 6.1 Hz, H-7), 2.55 (2H, dd, J=13.6 Hz, H-7′), 2.19 (2H, s, H-8, 8′) ¹³C-NMR (DMSO-d₆, 100 MHz): δ166.6 (C-9″, 9″′), 149.3 (C-4″, 4″′), 147.9 (C-3″, 3″′), 147.4 (C-3, 3′), 145.0 (C-7″, 7″′), 144.6 (C-4, 4′), 130.8 (C-1, 1′), 125.5 (C-1″, 1″′), 123.1 (C-6″, 6″′), 120.9 (C-6, 6′), 115.5 (C-5″, 5″′), 115.2 (C-5, 5′), 114.4 (C-8″, 8″′), 112.8 (C-2, 2′), 111.2 (C-2″, 2″′), 64.8 (C-9, 9′), 55.6 (—OCH₃), 55.3 (—OCH₃), 39.10 (C-8, 8′), 34.7 (C-7, 7′)

(B) Compound 2: Platyphyllenone

appearance: yellow oil

Molecular formulae: C₁₉H₂₀O₃(MW. 296.14);

¹H NMR (CD₃OD, 500 MHz) δ: 6.98 (4H, m, aromatic protons), 6.87 (1H, d, J=15.9 Hz, H-5), 6.68 (4H, m, aromatic protons), 6.05 (1H, dt, J=15.9, 1.4 Hz, H-4), 2.78 (4H, m, H-1, 2), 2.66 (2H, t, J=7.5 Hz, H-6), 2.45 (2H, m, H-7).

¹³C NMR (CD₃OD, 125 MHz) δ: 202.8 (C-3), 156.69 (C-4′), 156.64 (C-4″), 149.22 (C-5), 133.18 (C-1′), 132.99 (C-1″), 131.64 (C-4), 130.37 (C-2′, 2″), 130.31 (C-6′, 6″), 116.17 (C-3′, 3″), 116.15 (C-5′, 5″), 42.7 (C-2), 35.7 (C-7), 34.5 (C-6), 30.6 (C-1).

(C) Compound 3: (5R)—O-Methylhirsutanonol

appearance: brown oil

Molecular formulae: C₂₀H₂₄O₆(MW. 360.16);

¹H NMR (CD₃OD, 500 MHz) δ: 6.67 (2H, dd, J=7.9, 4.8 Hz, aromatic proton), 6.62 (2H, d, J=2.9 Hz, aromatic protons), 6.50 (2H, dd, J=9.7, 3.8 Hz, aromatic protons), 3.65 (1H, dt, J=12.0, 6.0 Hz, H-5), 3.28 (3H, s, —OCH₃), 2.68 (5H, m, H-1, 2), 2.50 (3H, m, H-7), 2.50 (1H, m, H-4), 1.71 (2H, m, H-6); ¹³C NMR (CD₃OD, 125 MHz) δ: 211.59 (C-3), 146.18 (C-4′), 146.15 (C-4″), 134.80 (C-1′), 134.02 (C-1″), 120.61, 120.56 (aromatic carbon), 116.51, 116.49, 116.34 (aromatic carbon), 77.93 (C—OCH₃), 57.06 (C-5), 48.25 (C-4), 46.35 (C-1), 36.97 (C-6), 31.63 (C-7), 30.10 (C-2).

(D) Compound 4: Hirsutanonol

appearance: yellow oil

Molecular formulae: C₁₉H₂₂O₆(MW. 346.14);

¹H NMR (CD₃OD, 500 MHz) δ: 6.65 (4H, m, aromatic proton), 6.51 (2H, ddd, J=8.0, 3.7, 2.0 Hz, aromatic protons), 4.02 (1H, dq, J=12.5, 6.3 Hz, H-3), 2.72 (4H, m, H-1, 2), 2.54 (4H, m, H-4, 7), 1.67 (2H, m, H-6); ¹³C NMR (CD₃OD, 125 MHz) δ: 212.03 (C-3), 146.17 (C-3′), 146.12 (C-3″), 144.46 (C-4′), 144.23 (C-4″), 134.94 (C-1′), 134.07 (C-1″), 120.64 (C-6′), 120.51 (C-6″), 116.54 (C-2′), 116.46 (C-5′), 116.34 (C-2″), 116.30 (C-5″), 68.31 (C-5), 51.26 (C-4), 46.37 (C-2), 40.44 (C-6), 32.17 (C-7) 30.04 (C-1).

(E) Compound 5: Platyphylloside

appearance: brown oil

Molecular formulae: C₂₅H₃₂O₉(MW. 476.52);

¹H NMR (CD₃OD, 500 MHz) δ: 7.02 (1H, t, J 8.5 Hz, aromatic protons), 6.69 (1H, dd, J=8.4, 0.8 Hz, aromatic protons), 4.30 (1H, d, J 7.8 Hz, H-1″′), 4.18 (1H, m, H-5), 3.88 (1H, dd, J=11.8, 2.3 Hz, H-6″′b), 3.72 (1H, dd, J=11.8, 5.3 Hz, H-6″′a), 3.36 (2H, dd, J=16.5, 8.6 Hz, H-3″′, 4″′,), 3.26 (1H, m, H-5″′), 3.16 (1H, t, J=8.4 Hz, H-2″′), 2.82 (1H, dd, J=16.6, 6.8 Hz, H-4b), 2.77 (4H, d, J=8.1 Hz, H-1, 2), 2.60 (3H, m, H-4a, 7), 1.85 (1H, ddd, J=13.6, 8.6, 6.9 Hz, H-6b), 1.75 (1H, m, H-6a); ¹³C NMR (CD₃OD, 125 MHz) δ: 211.88 (C-3), 156.58 (C-4′), 156.30 (C-4″), 134.33 (C-1″), 133.23 (C-1′), 130.43 (C-2, 2″), 130.33 (C-6′, 6″), 116.18 (C-3′, 3″), 116.05 (C-5′, 5″), 103.46 (C-1″′), 78.05 (C-3″′), 77.83, (C-5″′), 76.21 (C-5), 75.21 (C-2″′), 71.59 (C-4″′), 62.75 (C-6″′), 48.69 (C-4), 46.41 (C-2), 38.51 (C-6), 31.43 (C-7), 29.82 (C-1).

(F) Compound 6: Oregonin

appearance: brown powder

Molecular formulae: C₂₄H₃₀O₁₀ (MW 478.49)

¹H NMR (Me₂SO-d₆+D₂O, 500 MHz) δ: 6.71-6.75 (4H, H-2′, 2″, 5′, 5″), 6.52 (2H, dd, J=8.1, 2.1 Hz, H-6′, 6″), 4.32 (1H, d, J=7.8 Hz, xyl-1), 4.14 (1H, m, H-5), 3.90 (1H, dd, J=11.4, 6.1 Hz, xyl-5 eq), 3.56 (1H, m, xyl-4), 3.48 (1H, dd, J=9.0, 9.0 Hz, xyl-3), 3.23 (1H, dd, J=11.4, 11.2 Hz, xyl-5a), 3.21 (1H, dd, J=9.0, 7.8 Hz, xyl-2), 2.52-2.86 (8H, H-1, 4, 6, 7), 1.75-1.82 (2H, m, H-2). ¹³C NMR (Me₂SO-d₆+D₂O, 125 MHz) δ: 211.0 (C-3), 145.7 (C-3″), 145.3 (C-3′), 144.1 (C-4″), 143.8 (C-4′), 134.8 (C-1″), 133.8 (C-1′), 120.4 (C-6″), 120.4 (C-6′), 116.4 (C-5″), 116.4 (C-5′), 116.2 (C-2″), 116.1 (C-2′), 103.8 (xyl-1), 77.3 (xyl-3), 76.1 (C-5), 74.5 (xyl-2), 70.6 (xyl-4), 66.4 (xyl-5), 48.1 (C-4), 46.0 (C-2), 38.1 (C-6), 31.3 (C-7), 29.2 (C-1).

### Experimental Example 1. Stimulation Effect of Inventive Extract on the Initiation Effect of Myoblast Differentiation

In order to investigate the myogenic effect of the inventive extract obtained in Examples on the myoblast differentiation, following experiment was performed according to the procedure disclosed in the previous literature (Chem. Biol. Interact. 2016, 248:60).

1-1. Test Procedure

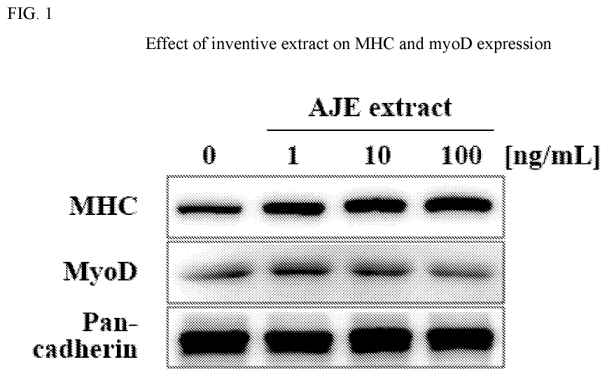

Various concentrations of inventive extract (1, 10, 100 ng/ml) were treated to C2C12 cells (mouse myoblast cell line; Ca^{#}CRL-1771, American Type Culture Collection (ATCC)) and cell lysates obtained from the differentiated cells, were subjected to Western Blot analysis to evaluate the expressed level of MHC and myoD proteins (The result was shown in FIG. 1A). the change of MHC expression caused by inventive extract in myotube cell was determined by immunostaining method using by mouse anti-MHC and anti-mouse IgG2b Alexa-fluor 568 (See FIG. 1B) (Chem. Biol. Interact. 2016, 248:60).

Various concentrations of inventive extract (1, 10, 100 ng/ml) were treated to C2C12 cells (mouse myoblast cell line; Cat^{#}CRL-1771, ATCC) in differentiation medium (2% horse serum-containing DMEM, Cat^{#}11965-084, Gibco) for 3 days to induce their differentiation.

1-1-1. Western Blot Analysis

For Western blot analysis, about 3×10⁴ mouse myoblast cell cells were incubated in 60 mm plates for 24 hours, and each sample was added to the differentiation medium for 3 days to differentiate. The protein extract was obtained from the differentiated cells using by lysis buffer [25 mM Tris-HCl (pH 7.5), 100 mM NaCl, 1% NP-40, 1% sodium deoxycholate, 0.1% sodium dodecyl sulfate (SDS), and protease inhibitor cocktail, Calbiochem, Darmstadt, Germany). The protein extract (25 μg) was performed to SDS-polyacrylamide gel electorphoresis (PAGE) and transferred to polyvinylidene fluoride (PVDF) membranes.

Primary mouse anti-MHC (sc-376157, Santa Cruz) or anti-myoD (sc-32758, Santa Cruz) was conjugated to the blots at 4° C., for 12 hrs and then conjugated with anti-mouse secondary antibody conjugated to Horseradish peroxidase (goat anti-mouse IgG-HRP, sc-2005, SantaCruz) to analyze the amount of MHC protein by chemiluminescence method. Pan-cadherin (Cat #3678, Sigma) was used as a loading control in the experiment.

1-1-2. Immunofluorescence Staining

According to the above-described method, the differentiated myoblast cells in the differentiation medium were subject to immunofluorescence staining method.

After removing respective differentiation medium, the differentiated myoblast cells in the differentiation medium were washed with phosphate buffer solution twice and fixed with 4% paraformaldehyde (0141, BBC Biochemical) for 20 mins. After washing the cells with phosphate buffer solution twice again, the cells were treated with 0.1% tritonX-100 (2315025, Sigma) for 20 mins. After washing the cells with phosphate buffer solution twice again, the cells were blocked with 5% horse serum solution (16050122, Gibco) and mouse anti-MHC (MAB4470, R&D systems) was added thereto to react for 12 hours at 4° C.

After finishing the reaction, the cells were washed with phosphate buffer solution more than 3 times and then the level of MHC expression was analyzed using by Alexa Flouor 568-conjuagted secondary anti-mouse (A-21144, MicoProbes) and DAPI (D9542, Sigma). The test result of immunofluorescence on MHC-positive myotubes was visualized with red color and the DAPI-labelled nucleus was visualized with blue color.

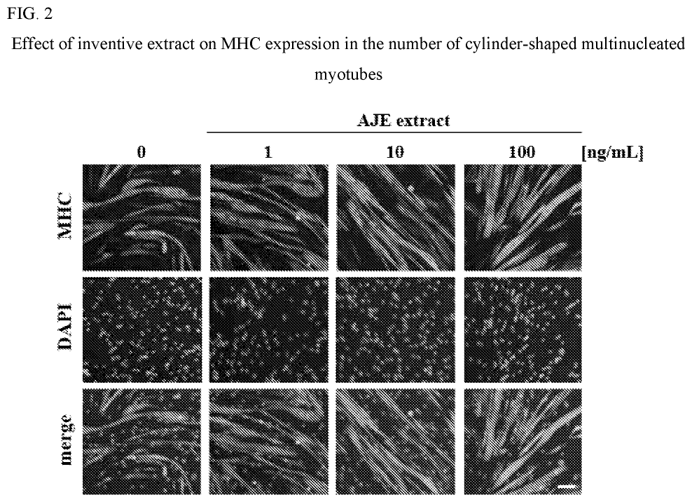

1-2. Test Result (See Table 1 and FIG. 1-2)

As shown in FIG. 1, the effect of test sample on the expression of MHC and myoD was determined. The cells added with various concentrations of inventive extract (1, 10, 100 ng/ml) were treated to myotubes differentiation medium to be differentiate to muscle fiber for 3 days. The muscle fibers were collected and the level of differentiation markers to muscle fibers, i.e., MHC and myoD protein was analyzed according to Western blot analysis.

At the result, it can be confirmed that the test sample group treated with inventive extract showed increased expression level comparing with the control group of which expression level of MHC and myoD was set to 1. (See Table 1).

As shown in FIG. 2, the effect of test sample on the muscle differentiation was determined according to immunofluorescence staining method using by anti-MHC antibody and DAPI (4′,6-diamidino-2-phenylindole). The cells added with various concentrations of inventive extract (1, 10, 100 ng/ml) were treated to myotubes differentiation medium to be differentiate to muscle fiber for 3 days. The muscle fibers were collected and the level of differentiation markers to muscle fibers, i.e., MHC and myoD protein was analyzed according to Western blot analysis.

Increased red-fluorescence means that the inventive extract promotes MHC expression in C2C12 cells in a concentration-dependent manner, and it has been confirmed that cylinder-shaped myoblast cells expressing MHC are found to be multinucleated through DAPI counter staining method (See Table 2).

In this experiment, it has been confirmed that inventive extract increases the expression of MHC and myoD protein in myotube cells and the number of cylinder-shaped multinucleated myotubes, resulting in promoting muscle cell differentiation (See Table 1-2, FIG. 1-2).

TABLE 1
Effect of inventive extract on the expression
of MHC and myoD (See FIG. 1)

AJE (ng/mL)

| concentration | 0 | 1 | 10 | 100 |
|---|---|---|---|---|
| MHC/pan-cadherin | 1.0 | 1.3 | 1.4 | 1.6 |
| expression (fold) | | | | |
| myoD/pan-cadherin | 1.0 | 1.8 | 1.7 | 1.5 |
| expression (fold) | | | | |

TABLE 2
Effect of inventive extract on the expression of MHC in
cylinder-shaped multinucleated myotubes (See FIG. 2)

| | AJE (ng/mL) | | | |
|---|---|---|---|---|
| concentration | 0 | 1 | 10 | 100 |
| The number of muscle fibers that are | 1 | 3.3 | 5.2 | 5.2 |
| multinucleated with five or more | | | | |
| nuclei and expressed with MHC (fold) | | | | |

### Experimental Example 2. Study on p38 MAPK Signaling System Mechanism of Inventive Extract for Promoting Myoblast Differentiation

In order to determine the effect of the inventive extract obtained in Examples on p38 MAPK signaling system mechanism for promoting myoblast differentiation, following experiment was performed according to the procedure disclosed in the literature (Mol. Biol. Cell. 2010, 21:2399; Trends Cell Biol. 2006, 16:36).

Following test has been performed according to the procedure disclosed in the literature to confirm whether the inventive extract takes an effect to p38 MAPK signaling system mechanism during myoblast differentiation or not since it has been reported that p38 MAPK signaling system mechanism is very important to promote myoblast differentiation (See FIG. 2) (Mol. Biol. Cell. 2010, 21:2399).

p38 mitogen-activated protein kinase (p38 MAPK) plays an important role in MyoD activation, and p38MAP promotes the dimerization of MyoD to induce the expression of myogenic factors (Trends Cell Biol. 2006, 16:36).

2-1. Test Procedure

Various concentrations of inventive extract (1, 10, 100 ng/ml) were treated to myoblast cells and the expressed level of p38 MAPK protein in the lysates of differentiated myotubes obtained at 3 days after the differentiation, was determined according to Western blot analysis.

Primary rabbit-anti phosphorylation p38 MAPK (Cat #9212, Cell Signaling Technology) was reacted with anti-rabbit secondary antibody (goat anti-rabbit IgG-HRP, sc-2004, SantaCruz) to determine the express level of phosphorylated p38 MAPK and primary rabbit-anti-p38 MAPK (Cat #9212, Cell Signaling Technology) was used to determine the expressed amount of total p38 MAPK (loading control)

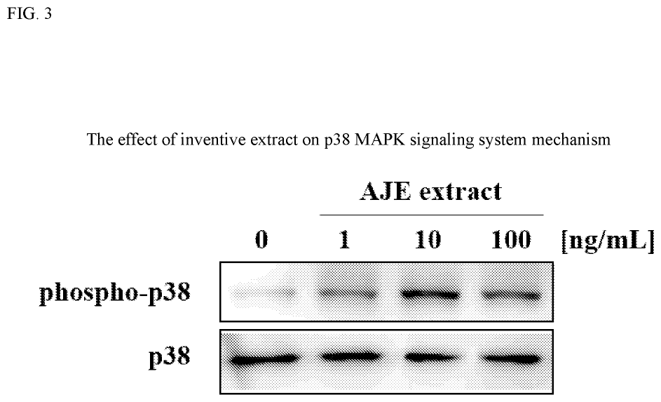

2-2. Test Result (Table 3 and FIG. 3)

It has been confirmed that p38 MAPK signaling system, which is important for myoblast cell differentiation, was further activated during differentiation by the treatment of inventive samples through Western assays which confirms the promoting effect of inventive extract on p38 MAPK (See Table 3).

The above test result confirm that the inventive extract promotes the myoblast differentiation through p38 MAPK activation mechanism.

TABLE 3
Effect of inventive extract on p38 MAPK activity (FIG. 3)

AJE (ng/mL)

| Concentration | 0 | 1 | 10 | 100 |
|---|---|---|---|---|
| phosphorylated-p38/p38 | 1.0 | 5.9 | 9.0 | 5.7 |
| expressed amount (fold) | | | | |

### Experimental Example 3. Stimulation Effect of Inventive Compounds on the Initiation Effect of Myoblast Differentiation

In order to investigate the myogenic effect of the inventive compounds 1-6 obtained in Examples on the myoblast differentiation, following experiment was performed according to the procedure disclosed in the previous literature (Chem. Biol. Interact. 2016, 248:60).

3-1. Test Procedure

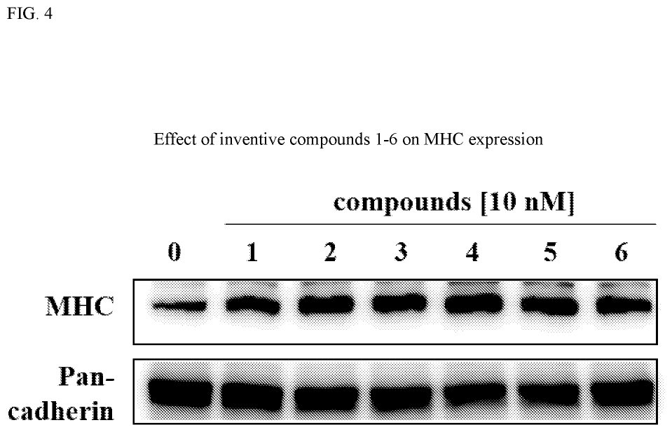

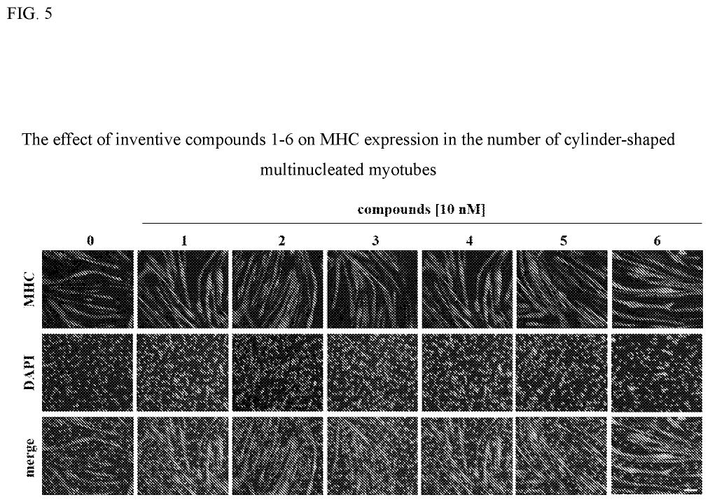

Various inventive compounds (10 nM) were treated to C2C12 cells (mouse myoblast cell line; Cat #CRL-1771, American Type Culture Collection (ATCC)) and cell lysates obtained from the differentiated cells, were subjected to Western Blot analysis to evaluate the expressed level of MHC proteins (The result was shown in FIG. 4). the change of MHC expression caused by inventive compounds in myotube cell was determined by immunostaining method using by mouse anti-MHC and anti-mouse IgG2b Alexa-fluor 568 (See FIG. 5) (Chem. Biol. Interact. 2016, 248:60).

Various inventive compounds (10 nM) were treated to C2C12 cells (mouse myoblast cell line; Cat^{#}CRL-1771, ATCC) in differentiation medium (2% horse serum-containing DMEM, Cat^{#}11965-084, Gibco) for 3 days to induce their differentiation.

3-1-1. Western Blot Analysis

For Western blot analysis, about 3×10⁴ mouse myoblast cell cells were incubated in 60 mm plates for 24 hours, and each sample was added to the differentiation medium for 3 days to differentiate. The protein extract was obtained from the differentiated cells using by lysis buffer [25 mM Tris-HCl (pH 7.5), 100 mM NaCl, 1% NP-40, 1% sodium deoxycholate, 0.1% sodium dodecyl sulfate (SDS), and protease inhibitor cocktail, Calbiochem, Darmstadt, Germany). The protein extract (25 μg) was performed to SDS-polyacrylamide gel electrophoresis (PAGE) and transferred to polyvinylidene fluoride (PVDF) membranes.

Primary mouse anti-MHC (sc-376157, Santa Cruz) or anti-myoD (sc-32758, Santa Cruz) was conjugated to the blots at 4° C., for 12 hrs and then conjugated with anti-mouse secondary antibody conjugated to Horseradish peroxidase (goat anti-mouse IgG-HRP, sc-2005, SantaCruz) to analyze the amount of MHC protein by chemiluminescence method. Pan-cadherin (Cat #3678, Sigma) was used as a loading control in the experiment.

3-1-2. Immunofluorescence Staining

According to the above-described method, the differentiated myoblast cells in the differentiation medium were subject to immunofluorescence staining method.

After removing respective differentiation medium, the differentiated myoblast cells in the differentiation medium were washed with phosphate buffer solution twice and fixed with 4% paraformaldehyde (0141, BBC Biochemical) for 20 mins. After washing the cells with phosphate buffer solution twice again, the cells were treated with 0.1% tritonX-100 (2315025, Sigma) for 20 mins. After washing the cells with phosphate buffer solution twice again, the cells were blocked with 5% horse serum solution (16050122, Gibco) and mouse anti-MHC (MAB4470, R&D systems) was added thereto to react for 12 hours at 4° C.

After finishing the reaction, the cells were washed with phosphate buffer solution more than 3 times and then the level of MHC expression was analyzed using by Alexa Flouor 568-conjuagted secondary anti-mouse (A-21144, MicoProbes) and DAPI (D9542, Sigma). The test result of immunofluorescence on MHC-positive myotubes was visualized with red color and the DAPI-labelled nucleus was visualized with blue color.

3-2. Test Result (See Table 4 and FIG. 4-5)

As shown in FIG. 4, the effect of test sample (inventive compounds 1-6) on the expression of MHC and myoD was determined. The cells added with various concentrations of inventive compounds (10 nM) were treated to myotubes differentiation medium to be differentiate to muscle fiber for 3 days. The muscle fibers were collected and the level of differentiation markers to muscle fibers, i.e., MHC protein was analyzed according to Western blot analysis.

At the result, it can be confirmed that the test sample group treated with inventive compounds showed increased expression level comparing with the control group of which expression level of MHC was set to 1. (See Table 4).

As shown in FIG. 5, the effect of test sample on the muscle differentiation was determined according to immunofluorescence staining method using by anti-MHC antibody and DAPI (4′,6-diamidino-2-phenylindole). The cells added with inventive compounds (10 nM) were treated to myotubes differentiation medium to be differentiate to muscle fiber for 3 days. The muscle fibers were collected and the level of differentiation markers to muscle fibers, i.e., MHC and myoD protein was analyzed according to Western blot analysis.

Increased red-fluorescence means that the inventive compounds promote MHC expression in C2C12 cells in a concentration-dependent manner, and it has been confirmed that cylinder-shaped myoblast cells expressing MHC are found to be multinucleated through DAPI counter staining method.

In this experiment, it has been confirmed that inventive compounds 1-6 increases the expression of MHC protein in myotube cells and the number of cylinder-shaped multinucleated myotubes, resulting in promoting muscle cell differentiation (See Table 4-5, FIG. 4-5).

TABLE 4
Effect of inventive compounds on the expression
of MHC and myoD (See FIG. 4)

| compound (10 nM) | — | 1 | 2 | 3 | 4 | 5 | 6 |
|---|---|---|---|---|---|---|---|
| MHC/pan-cadherin | 1.0 | 1.5 | 1.7 | 1.6 | 1.9 | 1.9 | 1.5 |
| expression (fold) | | | | | | | |

TABLE 5
Effect of inventive compounds on the expression of MHC
in cylinder-shaped multinucleated myotubes (See FIG. 5)

| Compound (10 nM) | — | 1 | 2 | 3 | 4 | 5 | 6 |
|---|---|---|---|---|---|---|---|
| The number of muscle fibers | 1.0 | 2.8 | 3.2 | 3.1 | 3.1 | 3.2 | 2.9 |
| that are multinucleated with | | | | | | | |
| five or more nuclei and | | | | | | | |
| expressed with MHC (fold) | | | | | | | |

### Experimental Example 4. Promoting Effect of Inventive Compound on Myoblast Differentiation

In order to investigate the promoting effect of the inventive compound 1 obtained in Examples on myoblast differentiation, following experiment was performed according to the procedure disclosed in the previous literature (Chem. Biol. Interact. 2016, 248:60).

4-1. Test Procedure

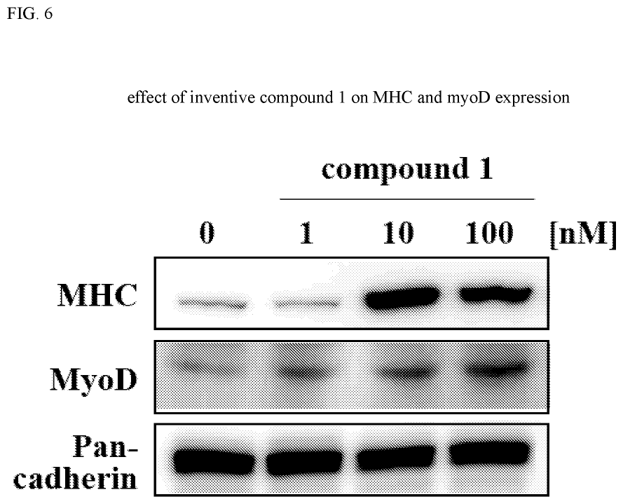

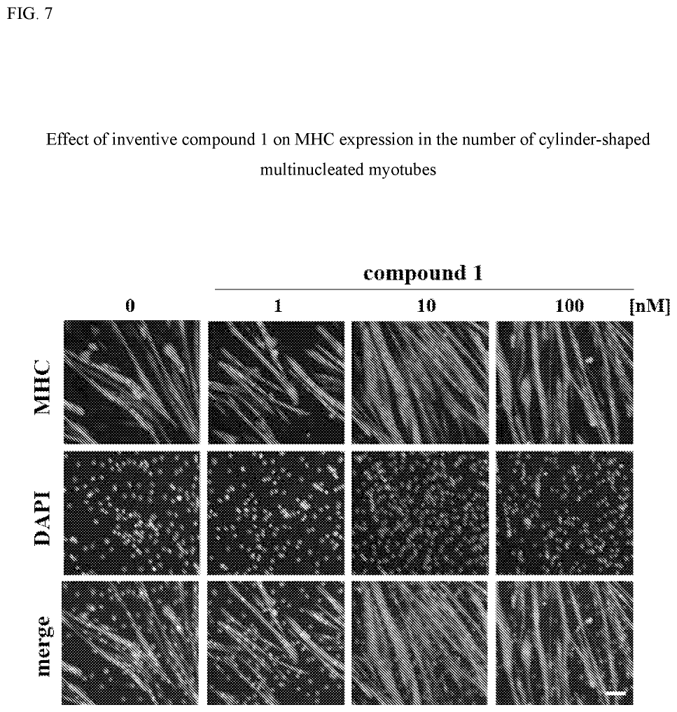

In order to investigate the myogenic effect of the inventive compound 1 obtained in Example, various concentrations of inventive compound 1 (1, 10 and 100 nM) were treated to C2C12 cells (mouse myoblast cell line; Cat^{#}CRL-1771, American Type Culture Collection (ATCC)) and cell lysates obtained from the differentiated cells, were subjected to Western Blot analysis to evaluate the expressed level of MHC and myoD proteins (The result was shown in FIG. 6). The change of MHC expression caused by inventive compound in myotube cell was determined by immunostaining method using by mouse anti-MHC and anti-mouse IgG2b Alexa-fluor 568 (See FIG. 7) (Chem. Biol. Interact. 2016, 248:60).

Various concentrations of inventive compound 1 (1, 10 and 100 nM) were treated to C2C12 cells (mouse myoblast cell line; Cat^{#}CRL-1771, ATCC) in differentiation medium (2% horse serum-containing DMEM, Cat^{#}11965-084, Gibco) for 3 days to induce their differentiation.

4-1-1. Western Blot Analysis

For Western blot analysis, about 3×10⁴ mouse myoblast cell cells were incubated in 60 mm plates for 24 hours, and each sample was added to the differentiation medium for 3 days to differentiate. The protein extract was obtained from the differentiated cells using by lysis buffer [25 mM Tris-HCl (pH 7.5), 100 mM NaCl, 1% NP-40, 1% sodium deoxycholate, 0.1% sodium dodecyl sulfate (SDS), and protease inhibitor cocktail, Calbiochem, Darmstadt, Germany). The protein extract (25 μg) was performed to SDS-polyacrylamide gel electorphoresis (PAGE) and transferred to polyvinylidene fluoride (PVDF) membranes.

Primary mouse anti-MHC (sc-376157, Santa Cruz) or anti-myoD (sc-32758, Santa Cruz) was conjugated to the blots at 4° C., for 12 hrs and then conjugated with anti-mouse secondary antibody conjugated to Horseradish peroxidase (goat anti-mouse IgG-HRP, sc-2005, SantaCruz) to analyze the amount of MHC protein by chemiluminescence method. Pan-cadherin (Cat #3678, Sigma) was used as a loading control in the experiment.

4-1-2. Immunofluorescence Staining

According to the above-described method, the differentiated myoblast cells in the differentiation medium were subject to immunofluorescence staining method.

After removing respective differentiation medium, the differentiated myoblast cells in the differentiation medium were washed with phosphate buffer solution twice and fixed with 4% paraformaldehyde (0141, BBC Biochemical) for 20 mins. After washing the cells with phosphate buffer solution twice again, the cells were treated with 0.1% tritonX-100 (2315025, Sigma) for 20 mins. After washing the cells with phosphate buffer solution twice again, the cells were blocked with 5% horse serum solution (16050122, Gibco) and mouse anti-MHC (MAB4470, R&D systems) was added thereto to react for 12 hours at 4° C.

After finishing the reaction, the cells were washed with phosphate buffer solution more than 3 times and then the level of MHC expression was analyzed using by Alexa Flouor 568-conjuagted secondary anti-mouse (A-21144, MicoProbes) and DAPI (D9542, Sigma). The test result of immunofluorescence on MHC-positive myotubes was visualized with red color and the DAPI-labelled nucleus was visualized with blue color.

4-2. Test Result (See Table 6 and FIG. 6-7)

As shown in FIG. 6, the effect of test sample (inventive compounds 1) on the expression of myoD was determined. The cells added with various concentrations of inventive compound 1 (1, 10, 100 nM) were treated to myotubes differentiation medium to be differentiate to muscle fiber for 3 days. The muscle fibers were collected and the level of differentiation markers to muscle fibers, i.e., MHC and myoD protein were analyzed according to Western blot analysis.

At the result, it can be confirmed that the test sample group treated with inventive compound 1 showed increased expression level comparing with the control group of which expression level of MHC and myoD was set to 1. (See Table 6).

As shown in FIG. 7, the effect of compound 1 on the muscle differentiation was determined according to immunofluorescence staining method using by anti-MHC antibody and DAPI (4′,6-diamidino-2-phenylindole).

Increased red-fluorescence means that the inventive compound 1 promotes MHC expression in C2C12 cells in a concentration-dependent manner, and it has been confirmed that cylinder-shaped myoblast cells expressing MHC are found to be multinucleated through DAPI counter staining method (See Table 7).

In this experiment, it has been confirmed that inventive compound 1 increases the expression of MHC and myoD proteins in myotube cells and the number of cylinder-shaped multinucleated myotubes, resulting in promoting muscle cell differentiation (See Table 6-7, FIG. 6-7).

TABLE 6
Effect of inventive compound 1 on the
expression of MHC and myoD (See FIG. 6)

compound 1(nM)

| concentration | 0 | 1 | 10 | 100 |
|---|---|---|---|---|
| MHC/pan-cadherin | 1.0 | 1.2 | 6.0 | 4.6 |
| expression (fold) | | | | |
| myoD/pan-cadherin | 1.0 | 1.2 | 1.3 | 1.7 |
| expression (fold) | | | | |

TABLE 7
Effect of inventive compound 1 on the expression of MHC
in cylinder-shaped multinucleated myotubes (See FIG. 7)

| | compound 1 (nM) | | | |
|---|---|---|---|---|
| concentration | 0 | 1 | 10 | 100 |
| The number of muscle fibers that are | 1 | 1.5 | 9.0 | 8.0 |
| multinucleated with five or more | | | | |
| nuclei and expressed with MHC (fold) | | | | |

### Experimental Example 5. Study on p38 MAPK Signaling System Mechanism of Inventive Compound 1 for Promoting Myoblast Differentiation

In order to determine the effect of the inventive compound 1 obtained in Examples on p38 MAPK signaling system mechanism for promoting myoblast differentiation, following experiment was performed according to the procedure disclosed in the literature (Mol. Biol. Cell. 2010, 21:2399; Trends Cell Biol. 2006, 16:36).

Following test has been performed according to the procedure disclosed in the literature to confirm whether the inventive compound 1 takes an effect to p38 MAPK signaling system mechanism during myoblast differentiation or not since it has been reported that p38 MAPK signaling system mechanism is very important to promote myoblast differentiation (Mol. Biol. Cell. 2010, 21:2399).

5-1. Test Procedure

Various concentrations of inventive compound 1 (1, 10, 100 nM) were treated to myoblast cells and the expressed level of p38 MAPK protein in the lysates of differentiated myotubes obtained at 3 days after the differentiation, was determined according to Western blot analysis.

Primary rabbit-anti phosphorylation p38 MAPK (Cat #9212, Cell Signaling Technology) was reacted with anti-rabbit secondary antibody (goat anti-rabbit IgG-HRP, sc-2004, SantaCruz) to determine the express level of phosphorylated p38 MAPK and primary rabbit-anti-p38 MAPK (Cat #9212, Cell Signaling Technology) was used to determine the expressed amount of total p38 MAPK (loading control)

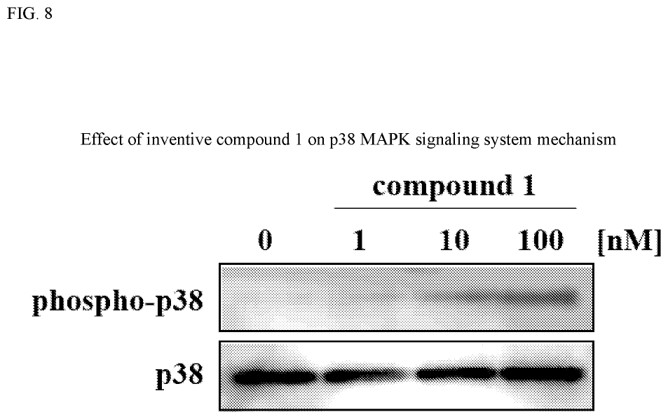

5-2. Test Result (Table 8 and FIG. 8)

It has been confirmed that p38 MAPK signaling system, which is important for myoblast cell differentiation, was further activated during differentiation by the treatment of inventive samples through Western assays which confirms the promoting effect of inventive compound 1 on p38 MAPK (See Table 8).

The above test result confirm that the inventive test sample promotes the myoblast differentiation through p38 MAPK activation mechanism.

TABLE 8
Effect of inventive compound 1 on p38 MAPK activity (FIG. 8)

Compound 1 (nM)

| Concentration | 0 | 1 | 10 | 100 |
|---|---|---|---|---|
| phosphorylated-p38/p38 | 1.0 | 10.6 | 14.8 | 15.0 |
| expressed amount (fold) | | | | |

### Experimental Example 6. Inhibitory Effect of Inventive Extract on Muscle Loss

In order to confirm the inhibitory effect on muscle loss of the inventive extract obtained in Examples, following experiment was performed according to the procedure disclosed in the previous literature (Int. J. Mol. Med. 2015, 36:29; Biomed. Pharmacother. 2017, 95:1486)

6-1. Test Procedure

In order to confirm the inhibitory effect of the inventive extract obtained in Example, dexamethasone was treated to myotube cell to establish in vitro model of muscle loss and following experiment was performed according to the procedure disclosed in the previous literature (Int. J. Mol. Med. 2015, 36:29).

In particular, following experiment was performed according to the procedure disclosed in the previous literature to find out whether inventive extract takes an effect on MAFbx expression during muscle protein degradation by dexamethasone treatment since the activity of MAFbx, a muscle proteolytic enzyme, is known to induce muscle protein loss (Biomed. Pharmacother. 2017, 95:1486)

6-1-1. Western Blot Analysis

For Western blot analysis, about 3×10⁴ mouse myoblast cell cells (C2C12; CRL-1772 ATCC) were incubated in 60 mm plates for 24 hours, and various concentrations of each inventive extract (0, 1, 10, 100 ng/mL) was added to the differentiation medium for 3 days to differentiate. The protein extract was obtained from the differentiated cells using by lysis buffer [25 mM Tris-HCl (pH 7.5), 100 mM NaCl, 1% NP-40, 1% sodium deoxycholate, 0.1% sodium dodecyl sulfate (SDS), and protease inhibitor cocktail, Calbiochem, Darmstadt, Germany). The protein extract (25 μg) was performed to SDS-polyacrylamide gel electorphoresis (PAGE) and transferred to polyvinylidene fluoride (PVDF) membranes.

Primary mouse anti-MHC (sc-376157, Santa Cruz) or primary mouse anti-MAFbx (sc-166806, Santa Cruz) was conjugated to the blots at 4° C., for 12 hrs and then conjugated with anti-mouse secondary antibody conjugated to Horseradish peroxidase (goat anti-mouse IgG-HRP, sc-2005, Santa Cruz) to analyze the amount of MHC and MAFbx protein by chemiluminescence method. Pan-cadherin (Cat #3678, Sigma) was used as a loading control in the experiment.

6-1-2. Immunofluorescence Staining

According to the above-described method, the differentiated myoblast cells in the differentiation medium were subject to immunofluorescence staining method.

After removing respective differentiation medium, the differentiated myoblast cells in the differentiation medium were washed with phosphate buffer solution twice and fixed with 4% paraformaldehyde (0141, BBC Biochemical) for 20 mins. After washing the cells with phosphate buffer solution twice again, the cells were treated with 0.1% tritonX-100 (2315025, Sigma) for 20 mins. After washing the cells with phosphate buffer solution twice again, the cells were blocked with 5% horse serum solution (16050122, Gibco) and mouse anti-MHC (MAB4470, R&D systems) was added thereto to react for 12 hours at 4° C.

After finishing the reaction, the cells were washed with phosphate buffer solution more than 3 times and then the level of MHC expression was analyzed using by Alexa Flouor 568-conjuagted secondary anti-mouse (A-21144, MicoProbes) and DAPI (D9542, Sigma). The test result of immunofluorescence on MHC-positive myotubes was visualized with red color and the DAPI-labelled nucleus was visualized with blue color.

6-2. Test Result

As a result of analyzing MHC protein expression in each cell group through Western blotting assays, when MHC expression in negative control group (NC), i.e., myotube cells that had not treated with dexamethasone, was set to 1, that in the positive control group, i.e., myotube cells treated with dexamethasone, showed the reduced level to 0.7 fold and that in test sample group i.e., myotube cells treated with inventive test sample, also showed recovered level.

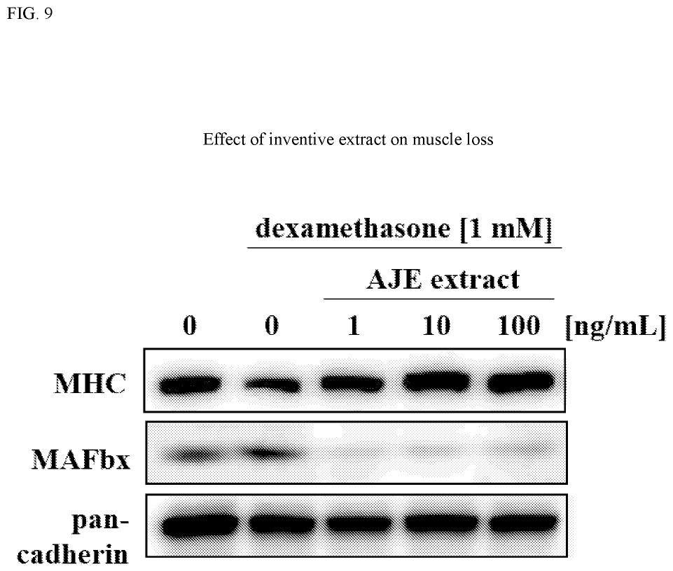

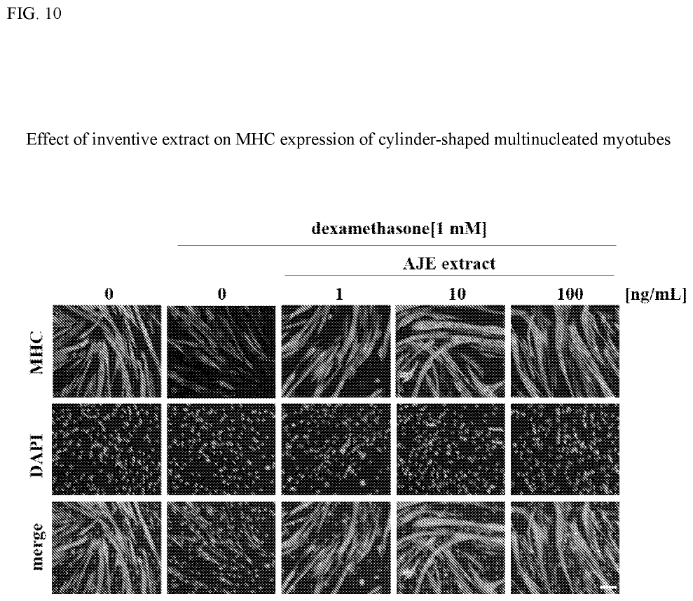

Additionally, it has been confirmed that when MAFbx expression in negative control group (NC), i.e., myotube cells that had not treated with dexamethasone, was set to 1, that in the positive control group, i.e., myotube cells treated with dexamethasone to indue muscle loss, showed the increased level to 1.9 fold, and the level had been decreased to 0.3 fold in a dose dependent manner in test sample group i.e., myotube cells treated with inventive test sample, which means the inhibitory effect of inventive extract on proteolysis of myoprotein (See Table 9 and FIG. 9-10).

As shown in FIG. 9, MHC expression was evaluated at the level of red fluorescence in myotube cells to which both of inventive test sample and dexamethasone were added, and the formation of multinucleated myotubes induced by treatment with inventive test samples was determined using by DAPI reading staining method.

It has been confirmed that the group treated with dexamethasone showed increased loss of myotube cell whereas the test group treated with test sample strong inhibitory effect on the loss of multinucleated MHC-positive cells caused by dexamethasone treatment (Tables 10 and FIG. 10).

Accordingly, it has been demonstrated that the inventive test sample strong protective activity from muscle loss.

TABLE 9
protecting activity of inventive extract on myotube cells (FIG. 9)

| | |
|---|---|
| Control | AJE (ng/mL) |

| | group | 0 | 1 | 10 | 100 |
|---|---|---|---|---|---|
| MHC/pan-cadherin | 1.0 | 0.9 | 1.3 | 1.4 | 1.6 |
| expression (fold) | | | | | |
| MAFbx/pan-cadherin | 1.0 | 1.9 | 0.3 | 0.3 | 0.6 |
| expression (fold) | | | | | |

TABLE 10
Effect of inventive extract on the expression of MHC in
cylinder-shaped multinucleated myotubes (See FIG. 10)

| | Control | AJE (ng/mL) | | | |
|---|---|---|---|---|---|
| | group | 0 | 1 | 10 | 100 |
| The number of muscle fibers that are | 1.0 | 0.1 | 0.7 | 0.9 | 0.8 |
| multinucleated with five or more | | | | | |
| nuclei and expressed with MHC (fold) | | | | | |

### Experimental Example 7. Inhibitory Effect of Inventive Compounds on Muscle Loss

In order to confirm the inhibitory effect on muscle loss of the inventive compounds obtained in Examples, following experiment was performed according to the procedure disclosed in the previous literature (Int. J. Mol. Med. 2015, 36:29; Biomed. Pharmacother. 2017, 95:1486)

7-1. Test Procedure

In order to confirm the inhibitory effect of the inventive compounds obtained in Example, dexamethasone was treated to myotube cell to establish in vitro model of muscle loss and following experiment was performed according to the procedure disclosed in the previous literature (Int. J. Mol. Med. 2015, 36:29).

About 3×10⁴ mouse myoblast cell cells (C2C12; CRL-1772 ATCC) were incubated in 60 mm plates for 24 hours, and various inventive compounds (10 nM) were added to the differentiation medium (0273, Gibco) for 3 days to differentiate.

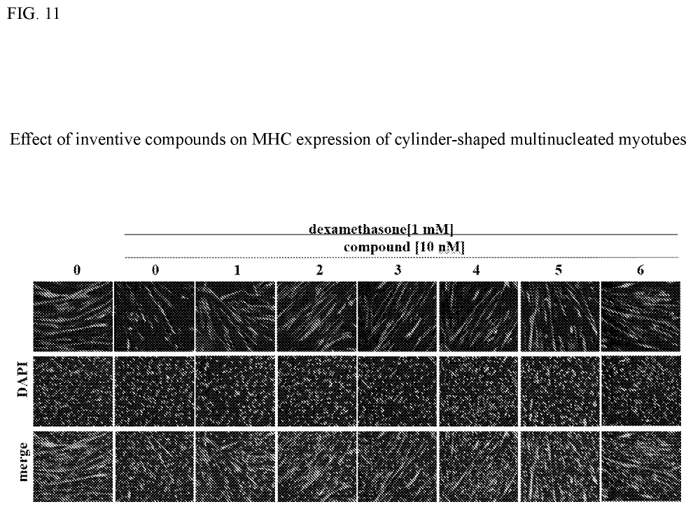

In order to establish in vitro model of muscle loss, 1 mM dexamethasone (D4902, Sigma) was treated to each differentiated myotube cell for 12 hours to differentiate and the cell was washed with phosphate buffer solution to perform immunofluorescence staining in order to confirm MHC expression (See FIG. 11).

7-1-1. Immunofluorescence Staining

According to the above-described method, the differentiated myoblast cells in the differentiation medium were subject to immunofluorescence staining method.

After removing respective differentiation medium, the differentiated myoblast cells in the differentiation medium were washed with phosphate buffer solution twice and fixed with 4% paraformaldehyde (0141, BBC Biochemical) for 20 mins. After washing the cells with phosphate buffer solution twice again, the cells were treated with 0.1% tritonX-100 (2315025, Sigma) for 20 mins. After washing the cells with phosphate buffer solution twice again, the cells were blocked with 5% horse serum solution (16050122, Gibco) and mouse anti-MHC (MAB4470, R&D systems) was added thereto to react for 12 hours at 4° C.

After finishing the reaction, the cells were washed with phosphate buffer solution more than 3 times and then the level of MHC expression was analyzed using by Alexa Flouor 568-conjuagted secondary anti-mouse (A-21144, MicoProbes) and DAPI (D9542, Sigma). The test result of immunofluorescence on MHC-positive myotubes was visualized with red color and the DAPI-labelled nucleus was visualized with blue color.

7-2. Test Result

The MHC expression was evaluated at the level of red fluorescence in myotube cells to which both of inventive test sample and dexamethasone were added, and the formation of multinucleated myotubes induced by treatment with inventive test samples was determined using by DAPI reading staining method.

It has been confirmed that the group treated with dexamethasone showed increased loss of myotube cell whereas the test group treated with test sample strong inhibitory effect on the loss of multinucleated MHC-positive cells caused by dexamethasone treatment (Tables 11 and FIG. 11).

Accordingly, it has been demonstrated that the inventive test sample strong protective activity from muscle loss.

TABLE 11
Effect of inventive compounds on the expression of MHC
in cylinder-shaped multinucleated myotubes in dexamethasone-
induced loss of myotube cell (See FIG. 11)

| | Control | compound (10 nM) | | | | | | |
|---|---|---|---|---|---|---|---|---|
| | group | 0 | 1 | 2 | 3 | 4 | 5 | 6 |
| The number of muscle fibers | 1.0 | 0.4 | 0.7 | 0.8 | 0.5 | 0.7 | 0.8 | 0.7 |
| that are multinucleated with | | | | | | | | |
| five or more nuclei and | | | | | | | | |
| expressed with MHC (fold) | | | | | | | | |

### Experimental Example 8. Improving Effect of Inventive Extract on Motility in Animal Model of Muscle Loss (In Vivo)

In order to confirm the improving effect on motility in animal model of muscle loss of the inventive compounds obtained in Examples, following experiment was performed according to the procedure disclosed in the previous literature (Int. J. Mol. Med. 2015, 36:29; Biomed. Pharmacother. 2017, 95:1486)

Dexamethasone, a synthetic glucocorticoid, was injected subcutaneously into C57/BL6 mouse mice to establish an animal model of muscle loss and following experiment was performed.

8-1. Test Procedure

In order to confirm the inhibitory effect of the inventive compounds obtained in Example, dexamethasone

To establish an in vivo model of muscle loss, 6 weeks-old male C57/BL6 mice (Raon Bio, 6 weeks old, weight range: 32-36 g) were acclimated for one-week, and then weighed animals determined to be healthy during the acclimatization period. The mice close to the average weight were selected using a simple random sampling method and were classified into five groups, namely, (a) a control group, (b) a muscle loss control group (treated with dexamethasone 1 mg/kg), (c) test sample group treated with inventive extract 250 mg/kg and dexamethasone, (d) test sample group treated with inventive extract 500 mg/kg and dexamethasone, and (e) positive control treated with oxymetholone (Celltrion Co. Ltd, ATC #A14AA05, 10 mg/kg) and dexamethasone.

The inventive Extract and oxymetholone were administered once to the mice daily for 4 weeks and dexamethasone was injected subcutaneously for 10 days from 10 days before the end of co-treatment of inventive extract and oxymetholone.

Both of inventive extract and oxymetholone were forcibly administered orally using zonde for oral administration after holding and fixing the skin of the animal's poll area, and dexamethasone was injected slowly subcutaneously after inserting a syringe into the dorsal skin fold of the animal held on the thumb and index finger, slightly aspirating whether it was injected normally, and confirming that no blood came out.

8-1-1. Determination of Body Weight

The body weight of every animal was measured once a week for 4 weeks from the day of inventive extract administration.

8-1-2. Grip Strength Test

The grip strength of mice was determined as follows; after grabbing the animal's tail and placing it on the grid, the front foot and body were pulled at the same speed until the front foot and body were held at the grid with the front paw, and the average value was calculated after three experimental repetitions. The determination was performed on the 14th and 28th days after the administration of test sample.

8-1-3. Treadmill Test

The treadmill test of mice was determined as follows; the speed of treadmill was set to 12 m/min as a starting speed, and the speed was gradually increased by 3 m per minute to reach to 30 m/min as a final speed, and the test was measured for a total of 30 minutes. The determination was performed on the 14th and 28th days after the administration of test sample.

8-1-4. Measurement of Muscle Mass, Epididymal Fat, and Liver Weight in Animal

The treadmill test of mice was determined as follows; the speed of treadmill was set to 12 m/min as a starting speed, and the speed was gradually increased by 3 m per minute to reach to 30 m/min. The mice were anesthetized with anesthetic to prevent the movement of experimental animals and sacrificed to minimize pain to dissect them. All instruments used during the experiment were sterilized and disinfected and their liver and epididymis fat were harvested.

The gastrocnemius muscle and soleus muscle of the mice were separated, washed in saline, drained, and weighed.

8-2. Test Result (Tables 12-16)

In order to confirm the inhibitory effect of the inventive test samples obtained in Example, dexamethasone

8-2-1. Change in Body Weight

Compared to the control group, there was no change in body weight in the sole treatment group with dexamethasone alone, and the group treated with inventive extract with 250 mg/kg and 500 mg/kg showed significant weight loss compared to the control group (See Table 12).

8-2-2. Motility Improving Activity

At the test result of grip strength measurements at week 4, it has been confirmed that the control group treated with dexamethasone showed decreased grip strength whereas the test group treated with 250 mg/kg and 500 mg/kg of inventive extract and positive control group showed significantly increased grip strength, which indicates that the inventive extract has the potential to improve muscle strength loss caused by muscular dystrophy. (See Table 12)

At the test result of 4 treadmill assessment at week 4, it has been confirmed that the group treated with dexamethasone showed decreased motility compared to the control group, and both of the test group treated with inventive extract and positive control group showed a tendency to increase motility reduced by dexamethasone treatment, of which difference was not significant. (See Table 14).

In summary, it has been confirmed that the inventive extract has the possibility to improve endurance-related motility activity.

8-2-3. Absolute Change in the Weight of Muscle Mass, Epididymal Fat, and Liver Weight in Animal (Table 15)

After autopsy at 4 weeks after administration, it has been confirmed that the groups showed no significant difference between the groups in respect of the weight of liver and epididymis fat while the test group treated with inventive extract (500 mg/kg) and positive control group showed significant decrease compared to dexamethasone treatment group.

As a result of the weighing of calf muscle, there was a significant decrease in the dexamethasone treatment group compared to the control group, while there was no significant change in either the test sample group treated with inventive extract or the positive control group compared to the dexamethasone treatment group.

As a result of the weighing of fluke muscle, the group treated with dexamethasone showed significant decrease compared to the control group, while both of test sample group treated with inventive extract 250 mg/kg or 500 mg/kg and positive control group showed significant increase compared to the dexamethasone treatment group.

As a result, it was confirmed that muscle loss by dexamethasone significantly can be increased by the treatment of inventive extract in a dose dependent manner.

8-2-4. Relative Change in the Weight of Muscle Mass, Epididymal Fat, and Liver Weight in Animal (Table 16)

After autopsy at 4 weeks after administration, the weight of liver and Epididymis fat was weighed to calculate weight-to-weight to compare the relative differences between the organ's weight in the groups.

It has been confirmed that the groups showed no significant difference between the groups in respect of the weight of liver and epididymis fat.

As a result of the weighing of calf muscle, there was a significant decrease in the dexamethasone treatment group compared to the control group, while there was significant increase in the test sample group treated with inventive extract (500 mg/kg) compared to the dexamethasone treatment group.

As a result of the weighing of fluke muscle, the group treated with dexamethasone showed significant decrease compared to the control group, while both of test sample group treated with inventive extract 250 mg/kg or 500 mg/kg and positive control group showed significant increase compared to the dexamethasone treatment group.

As a result, it was confirmed that muscle loss by dexamethasone significantly can be increased by the treatment of inventive extract in a dose dependent manner.

In summary, it has been confirmed that the inventive extract has the improving activity of motor capacity decline and muscle loss due to muscular atrophy under this test condition.

TABLE 12
Effect of inventive extract on the change in body weight (unit: gram)

| groups | 0 week | 1 week | 2 weeks | 3 weeks | 4 weeks |
|---|---|---|---|---|---|
| Control | 22.47 ± 0.82 | 23.17 ± 1.10 | 23.78 ± 1.18 | 24.08 ± 1.16 | 24.46 ± 0.98^{a} |
| Dexa | 22.33 ± 1.11 | 22.84 ± 1.54 | 23.46 ± 1.60 | 23.57 ± 1.81 | 23.57 ± 1.76^{ab} |
| Dexa + 250 | 22.31 ± 1.04 | 22.28 ± 0.82 | 22.70 ± 0.71 | 22.94 ± 0.93 | 22.74 ± 0.92^{b} |
| Dexa + 500 | 22.33 ± 1.10 | 22.51 ± 1.44 | 23.23 ± 1.07 | 22.60 ± 1.04 | 22.57 ± 1.29^{b} |
| Dexa + Oxy | 22.26 ± 0.71 | 22.49 ± 0.72 | 23.25 ± 0.90 | 23.22 ± 0.93 | 23.20 ± 0.78^{b} |

^{a, b}significance between the different characters (p < 0.01)
Dexa: dexamethasone treatment group
Oxy: oxymetholone

TABLE 13
Effect of inventive extract on the change in grip strength (unit: N)

| groups | 2 weeks | 4 weeks |
|---|---|---|
| Control | 1.09 ± 0.15 | 1.23 ± 0.12^{a} |
| Dexa | 1.05 ± 0.08 | 0.94 ± 0.12^{b} |
| Dexa + 250 | 1.01 ± 0.10 | 1.08 ± 0.11^{c} |
| Dexa + 500 | 1.04 ± 0.12 | 1.26 ± 0.07^{a} |
| Dexa + Oxy | 1.00 ± 0.06 | 1.25 ± 0.16^{a} |

^{a, b, c}significance between the different characters (p < 0.01)
Dexa: dexamethasone treatment group
Oxy: oxymetholone

TABLE 14
Effect of inventive extract on the motility (unit: meter)

| groups | 2 weeks | 4 weeks |
|---|---|---|
| Control | 776.98 ± 49.08 | 784.83 ± 38.13 |
| Dexa | 798.85 ± 14.33 | 745.73 ± 30.01* |
| Dexa + 250 | 793.93 ± 34.29 | 763.40 ± 37.72 |
| Dexa + 500 | 802.38 ± 27.62 | 783.55 ± 26.28 |
| Dexa + Oxy | 774.75 ± 66.11 | 773.93 ± 39.64 |

*significance compared to control group (p < 0.01)
Dexa: dexamethasone treatment group
Oxy: oxymetholone

TABLE 15
absolute change in the weight of muscle mass, epididymal
fat, and liver weight in animal (unit: gram)

| groups | liver | epididymal fat | calf muscle | fluke muscle |
|---|---|---|---|---|
| Control | 1.16 ± 0.11 | 0.50 ± 0.09 | 0.270 ± 0.019 | 0.019 ± 0.002^{ab} |
| Dexa | 1.15 ± 0.13 | 0.51 ± 0.10 | 0.242 ± 0.014* | 0.014 ± 0.002^{c} |
| Dexa + 250 | 1.11 ± 0.08 | 0.49 ± 0.10 | 0.246 ± 0.010* | 0.017 ± 0.003^{b} |
| Dexa + 500 | 1.10 ± 0.09 | 0.42 ± 0.05^{#} | 0.248 ± 0.007* | 0.020 ± 0.002^{a} |
| Dexa + Oxy | 1.16 ± 0.07 | 0.43 ± 0.09^{#} | 0.251 ± 0.016* | 0.023 ± 0.002^{d} |

*significance compared to control group (p < 0.01),
^{#}significance compared to dexamethasone treatment group (p < 0.01)
^{a, b, c, d}significance between the different characters (p < 0.01)
Dexa: dexamethasone treatment group
Oxy: oxymetholone

TABLE 16
relative change in the weight of muscle mass, epididymal
fat, and liver weight in animal (unit: gram)

| groups | liver | epididymal fat | calf muscle | fluke muscle |
|---|---|---|---|---|
| Control | 4.74 ± 0.33 | 2.04 ± 0.35 | 1.105 ± 0.070 | 0.077 ± 0.008^{a} |
| Dexa | 4.88 ± 0.33 | 2.17 ± 0.30 | 1.031 ± 0.072* | 0.060 ± 0.010^{b} |
| Dexa + 250 | 4.89 ± 0.28 | 2.17 ± 0.42 | 1.083 ± 0.043 | 0.075 ± 0.011^{a} |
| Dexa + 500 | 4.89 ± 0.34 | 1.86 ± 0.17 | 1.104 ± 0.068^{#} | 0.091 ± 0.012^{c} |
| Dexa + Oxy | 4.99 ± 0.29 | 1.86 ± 0.40 | 1.082 ± 0.061 | 0.099 ± 0.011^{c} |

*significance compared to control group (p < 0.01),
^{#}significance compared to dexamethasone treatment group (p < 0.01)
^{a, b, c}significance between the different characters (p < 0.01)
Dexa: dexamethasone treatment group
Oxy: oxymetholone

### MODE FOR INVENTION

Hereinafter, the formulating methods and kinds of excipients will be described, but the present invention is not limited to them. The representative preparation examples were described as follows.

### Preparation of Powder

Compound 5: 20 mg
Lactose: 100 mg
Talc: 10 mg

Powder preparation was prepared by mixing above components and filling sealed package.

### Preparation of Tablet

Compound 5: 10 mg
Corn Starch: 100 mg
Lactose: 100 mg
Magnesium stearate: 2 mg

Tablet preparation was prepared by mixing above components and entabletting.

### Preparation of Capsule

Compound 4: 10 mg
Crystalline cellulose: 3 mg
Lactose: 14.8 mg
Magnesium stearate: 0.2 mg

Tablet preparation was prepared by mixing above components and filling gelatin capsule by conventional gelatin preparation method.

### Preparation of Injection

Compound 3: 10 mg
mannitol: 180 mg
Distilled water for injection: 2974 mg
Na₂HPO₄, 12H2O: 26 mg

Injection preparation was prepared by dissolving active component, and then filling all the components in 2 ml ample and sterilizing by conventional injection preparation method.

### Preparation of Liquid

AJE extract: 20 mg
Isomerization sugar: 10 g
mannitol: 5 g
distilled water: optimum amount

Liquid preparation was prepared by dissolving active component, and then filling all the components in 100m ample and sterilizing by conventional liquid preparation method.

### Preparation of Health Food

AJE extract: 1000 mg
Vitamin mixture: optimum amount
Vitamin A acetate: 70 g
Vitamin E: 1.0 mg
Vitamin Bio: 13 mg
Vitamin B₂: 0.15 mg
Vitamin B6: 0.5 mg
Vitamin B1: 20.2 g
Vitamin C: 10 mg
Biotin: 10 g
Amide nicotinic acid: 1.7 mg
Folic acid: 50 g
Calcium pantothenic acid: 0.5 mg
Mineral mixture: optimum amount
Ferrous sulfate: 1.75 mg
Zinc oxide: 0.82 mg
Magnesium carbonate: 25.3 mg
Monopotassium phosphate: 15 mg
Dicalcium phosphate: 55 mg
Potassium citrate: 90 mg
Calcium carbonate: 100 mg
Magnesium chloride: 24.8 mg

The above mentioned vitamin and mineral mixture may be varied in many ways. Such variations are not to be regarded as a departure from the spirit and scope of the present invention.

### Preparation of Health Beverage

Compound 2: 1000 mg
Citric acid: 1000 mg
Oligosaccharide: 100 g
Apricot concentration: 2 g
Taurine: 1 g
Distilled water: 900 ml

Health beverage preparation was prepared by dissolving active component, mixing, stirred at 85° C. for 1 hour, filtered and then filling all the components in 1000m ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As mentioned the above, the inventors of the present invention confirmed the treating effect of an extract of alder tree or the compounds isolated therefrom mentioned above on skeleton muscle disease through various in vitro test and animal model tests, for example, stimulation effect of inventive extract on the initiation effect of myoblast differentiation (Experimental Example 1, 3 and 4), confirming that inventive extract/compounds increases the number of cylinder-shaped multinucleated myotubes, resulting in promoting muscle cell differentiation; Study on p38 MAPK signaling system mechanism of inventive extract/compounds for promoting myoblast differentiation (Experimental Example 2 and 5); Inhibitory effect of inventive extract/compounds on muscle loss (Experimental Example 6 and 7); improving effect on motility in animal model of muscle loss (In vivo) (Experimental Example 8) confirming that the inventive extract/compounds promotes muscle cell differentiation as well as improves motor capacity decline and muscle loss. Therefore, the inventive extract/compounds of the present invention can be usefully used in a pharmaceutical composition, health functional food, and health supplement food for preventing and treating on skeleton muscle disease.

1. A pharmaceutical composition comprising an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) as an active ingredient for the treatment or prevention of skeletal muscle diseases.

2. The pharmaceutical composition according to claim 1,
said alder tree is selected from Alnus japonica (Thunb.) Steudel or Alnus japonica var. koreana.

3. The pharmaceutical composition according to claim 1,
said alder tree is extracted from the group consisting of the root part, stem part, bark part, xylem part, herb part and leaf part of alder tree.

4. The pharmaceutical composition according to claim 1,
said extract of alder tree is selected from crude extract, non-polar solvent soluble extract or non-polar solvent soluble extract of alder tree.

5. The pharmaceutical composition according to claim 1,
said crude extract of alder tree is dissolved in polar solvent selected from distilled water, spirit, methanol, ethanol, butanol and the like, or the mixtures thereof.

6. The pharmaceutical composition according to claim 1,
said skeletal muscle disease is selected from the group consisting of muscular atony, muscular atrophy, muscular dystrophy, muscle degeneration, muscle stiffness, amyotrophic axonal sclerosis, myasthenia gravis, cachexia and sarcopenia.

7. (canceled)

8. (canceled)

9. A treating agent of cachexia or senile muscular atrophy comprising an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6) as an active ingredient.

10. (canceled)

11. (canceled)

12. (canceled)

13. (canceled)

14. (canceled)

15. A health functional food comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

16. The health functional food according to claim 15,
said health food is provided as powder, granule, tablet, chewing tablet, capsule or beverage type.

17. (canceled)

18. (canceled)

19. A health supplement food comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

20. (canceled)

21. (canceled)

22. A food additive comprising an extract of alder tree or the compounds isolated therefrom selected from (−)-(2R,3R)-1,4-O-diferuloylsecoisolariciresinol (DFS, compound 1}, platyphyllenone (compound 2), (5R)—O-methylhirsutanonol (compound 3), hirsutanonol (compound 4), platyphylloside (compound 5) or oregonin (compound 6) as an active ingredients for the improvement or prevention of skeletal muscle diseases.

23. (canceled)

24. (canceled)

25. A method of treating or preventing skeletal muscle diseases in a mammal suffering from skeletal muscle diseases comprising administering to said mammal an effective amount of an extract of alder tree or the compounds isolated therefrom selected from (−)-(2R,3R)-1,4-O-diferuloylsecoisolariciresinol (DFS, compound 1), platyphyllenone (compound 2), (5R)—O-methylhirsutanonol (compound 3), hirsutanonol (compound 4), platyphylloside (compound 5) or oregonin (compound 6).

26. (canceled)
